(19) United States
(12) Patent Application Publication
(10) Pub. No.: US 2026/0258223 A1
(43) Pub. Date: Sep. 3, 2026

(54) MATERIALS SHOWING ASYMETRIC RESPONSE TO MECHANICAL FORCE

(71) Applicant: RIKEN, Wako-shi, Saitama (JP)

(72) Inventors: Yasuhiro ISHIDA, Saitama (JP); Xiang WANG, Saitama (JP); Zhihao LI, Saitama (JP)

(73) Assignee: RIKEN, Wako-shi, Saitama (JP)

(21) Appl. No.: 19/159,758

(22) PCT Filed: Feb. 26, 2024
(86) PCT No.: PCT/JP2024/006755

§ 371 Date: Aug. 26, 2025

Related U.S. Application Data
(60) Provisional application No. 63/448,932, filed on Feb. 28, 2023.

(51) Int. Cl. C08K 7/00; C08F 220/56; C08J 3/21; C12N 5/07

(52) U.S. Cl. CPC C08K 7/00; C08F 220/56; C08J 3/212; C12N 5/0601; C08F 2810/20; C08J 2333/26; C08K 2201/01; C08K 2201/011; C12N 2537/10

(57) ABSTRACT

The present invention relates to a gel structure including a gel composition including at least one type of anisotropic nanostructures, at least one type of liquid, and at least one type of polymer having a crosslinked structure, which has at least one surface in a stationary state, and in which longitudinal axes of the at least one type of anisotropic nanostructures are oriented approximately parallel to each other and non-parallel and non-perpendicular to the at least one surface.

### TECHNICAL FIELD

The present invention relates to a material showing an asymmetric response to mechanical force.

### BACKGROUND ART

It is conceivable that a physical stimulus, such as an electric field, magnetic field, or light, is applied to a material along a single axis. In this situation, in most materials, stimuli are transmitted symmetrically when applied from right to left and when applied from left to right. However, in some materials, this symmetry is not maintained, and the output may differ depending on the direction of the stimulus applied; hereinafter, such a response will be referred to as an asymmetric response. Materials that exhibit such asymmetric responses are extremely useful in our daily lives because they can rectify and highly order disordered signals and energy. A typical example is a “diode,” which allows current to flow from right to left but does not allow current to flow from left to right.

There exist crystals and liquid crystals that exhibit functions derived from asymmetric structures (polar structures) in which molecules are assembled in asymmetric space groups. However, these can only show asymmetric responses to electrical, magnetic, and optical stimuli, and cannot show asymmetric responses to mechanical forces.

When solid materials are micromachined into asymmetric shapes (polar shapes) such as ratchet (sawtooth) or fishbone shapes, the entire workpiece exhibits an asymmetric response to mechanical forces macroscopically. However, this asymmetric response only holds when focusing on a size range larger than the structural unit (a single sawtooth or a single fishbone); when the size range is smaller, the response becomes symmetric rather than asymmetric.

There are many known materials that respond differently when the direction of the axis along which a mechanical force is applied is changed, i.e., they are anisotropic materials. However, this response is not an asymmetric response, and the mechanical signals and energy cannot be rectified or highly ordered.

### SUMMARY OF INVENTION

### Technical Problem

The present inventors have previously developed hydrogels containing various colloidal nanosheets (two-dimensional materials with a width of several micrometers and a thickness of 1 to 4 nanometers, which can be dispersed as colloids in water without overlapping). When synthesizing these hydrogels, the present inventors have also developed a technique to uniaxially orient the nanosheets in the gel in any direction by applying a strong magnetic field (approximately 10 Tesla).

While many materials are known to show an asymmetric response to electric fields, magnetic fields, light, or the like, no materials that show an asymmetric response to mechanical forces have been reported to date. The present inventors have now invented a hydrogel that exhibits an elastic modulus that differs more than 60 times between left shear and right shear. Hereinafter, this will be referred to as a “mechanically asymmetric gel.”

For instance, one example of the present invention is a hydrogel structure in which the nanosheets are oriented so as to tilt 45° clockwise from the direction of gravity when viewed from the side of the gel. When the bottom face of this hydrogel was fixed to a floor and a left or right shear force was applied to the top face, the difference in elastic modulus between both was 60 times or more, specifically as large as 67 times. This is because the left shear (nanosheets do not buckle) and the right shear (nanosheets buckle) are geometrically inequivalent. Thus, for the first time, a material showing an asymmetric response to mechanical force has been obtained.

### Solution to Problem

The gist of the present invention is as follows.
(1) A gel structure showing an asymmetric response to mechanical force.
(2) The gel structure according to (1), which is a hydrogel.
(3) The gel structure according to (2), wherein the hydrogel contains nanosheets.
(4) The gel structure according to (3), wherein the nanosheets are oriented so as to tilt 15° to 75° clockwise from a direction of gravity, as viewed from a side of the gel.
(5) The gel structure according to (3) or (4), wherein the nanosheets are graphene oxide nanosheets, niobium oxide nanosheets, titanium oxide nanosheets, clay nanosheets, alumina nanotubes, or carbon nanotubes.
(6) The gel structure according to any one of (1) to (5), wherein the mechanical force is a shear force or vibration.
(7) The gel structure according to (6), wherein when a bottom face of the gel structure is fixed to a floor and a left or right shear force is applied to a top face thereof, a difference in elastic modulus between both is 10 times or more.
(8) A method for producing the gel structure according to any one of (1) to (7), comprising changing a direction of a magnetic field such that the nanosheets are contained in a tilted state in the hydrogel.
(9) The method according to (8), comprising placing a pre-gel (gel precursor) solution including a monomer, a crosslinking agent, and graphene oxide nanosheets, niobium oxide nanosheets, titanium oxide nanosheets, clay nanosheets, alumina nanotubes, or carbon nanotubes in a container, and applying a magnetic field to the container while tilting the container, thereby polymerizing the monomer.
(10) The gel structure according to (1), which is a gel structure showing an asymmetric response to mechanical force, and comprises a gel composition including at least one type of anisotropic nanostructures, at least one type of liquid, and at least one type of polymer having a crosslinked structure.
(11) The gel structure according to (10), wherein the liquid is water.
(12) The gel structure according to (10), wherein the plurality of at least one type of anisotropic nanostructures is selected from the group consisting of graphene oxide nanosheets, niobium oxide nanosheets, titanium oxide nanosheets, clay nanosheets, alumina nanotubes, carbon nanotubes, and nanochannel pores.
(13) The gel structure according to (10) or (11), which has at least one surface in a stationary state, and in which longitudinal axes of the at least one type of anisotropic nanostructures are oriented approximately parallel to each other and non-parallel and non-perpendicular to the at least one surface.
(14) A gel structure comprising a gel composition including at least one type of anisotropic nanostructures, at least one type of liquid, and at least one type of polymer having a crosslinked structure, which has at least one surface in a stationary state, and in which longitudinal axes of the at least one type of anisotropic nanostructures are oriented approximately parallel to each other and non-parallel and non-perpendicular to the at least one surface.
(15) The gel structure according to any one of (10) to (14), which exhibits elastic moduli of different magnitudes when shear forces of the same magnitude are applied in a first direction and a second direction different from the first direction in a plane parallel to the one surface.
(16) The gel structure according to (10), which is in a film form.
(17) The gel structure according to (10), which is used as a culture medium for cells, microorganisms, or nematodes.
(18) A method for producing a gel structure, comprising:
   a first step of accommodating a gel raw material composition, which includes at least one type of anisotropic nanostructures, at least one type of liquid, and a polymerizable composition, in a container having at least one identifiable face; and
   a second step of carrying out a polymerization reaction of the polymerizable composition in the container,
   wherein in the second step, the at least one type of anisotropic nanostructures is oriented with their longitudinal axes approximately parallel to each other and non-parallel and non-perpendicular to the at least one identifiable face.
(19) The method according to (18), wherein in the second step, at least one type of physical stimulus is applied to the gel raw material composition.
(20) The method according to (18) or (19), wherein the polymerizable composition comprises at least one type of monomer, at least one type of crosslinking agent, and at least one type of polymerization initiator.

### Advantageous Effects of Invention

The gel structure of the present invention shows an asymmetric response and therefore exhibits the following interesting physical properties and functions.

Conversion of symmetric vibration to asymmetric vibration: When symmetric vibration is applied horizontally from the bottom of the gel structure of the present invention, it is transmitted to the top as asymmetric vibration. By utilizing this phenomenon, it is possible to convert the energy of random vibrations generated by a machine into unidirectional translational or rotational kinetic energy.

Asymmetric rebound of impacting object: When a small ball is free-dropped toward the gel structure of the present invention and impacts the surface perpendicularly, the small ball rebounds in a direction approximately 30° off from the angle of incidence. Since the difference between the angle of incidence and the angle of reflection can depend on the size, weight, and material of the sphere, the application of the gel structure of the present invention is expected for the separation of particles and powders. The application of the gel structure of the present invention as a highly functional impact-resistant material and in sporting goods is also expected.

Rectification of worm migration: The gel structure of the present invention can also be used as a culture medium. Specifically, it was confirmed that when worms were spread on the surface of this gel structure, all of them moved in the right direction. Control of worm migration direction has previously been achieved by creating gradients in light intensity, temperature, chemical concentration, and gel composition. However, the gel structure of the present invention is the only example of unidirectional migration of worms without relying on a gradient, and is expected to have great applications in cancer diagnosis using nematodes, which has become a hot topic in recent years. Furthermore, if similar control of migration direction could be achieved in cells (especially stem cells), it could have a huge impact on regenerative medical engineering. It can also be used to cultivate microorganisms.

### BRIEF DESCRIPTION OF DRAWINGS

FIG. 1A shows symmetric deformation of a 10 mm cube of the acrylamide hydrogel which is one example of a gel structure as a comparative example of the present invention. (i) Optical images. (ii) Relationship between the displacement and applied force.

FIG. 1B Upper of FIG. 1B shows one example of macroscopic buckling of a tilted beam connecting a fixed floor and a slidable ceiling. Lower of FIG. 1B shows microscopic buckling of a tilted nanofiller embedded in a matrix.

FIG. 1C shows schematic illustrations showing a design of one example of the mechanical asymmetric response gel structure of the present invention. FIG. 1C shows a schematic illustration of hydrogel as a matrix, a schematic illustration of unidirectionally oriented nanosheets of e.g., GO (graphene oxide) as nanofillers contained therein, and one design example of a non-reciprocal gel of one example of the gel structure of the present invention in which these are combined. The planes of the nanosheets include the y-axis of the x-y-z orthonormal coordinate, where the z-axis is parallel to gravity. The vector within the GO plane that is perpendicular to the y-axis is denoted as the plane indicator P (the same as the longitudinal axis of the nanosheets). The angle between the z-axis and P is denoted as the tilt angle θ.

FIG. 1D shows asymmetric deformation of the non-reciprocal gel structure which is one example (a 10 mm cube) of the gel structure of the present invention in response to shear forces applied at the top of the gel. (i) Optical images. (ii) Relationship between the displacement and applied force. The diagonal arrow in the middle of the top row is an indicator P of the GO plane in the gel matrix as defined in FIG. 1C.

FIG. 2A shows stress-strain curve of one example of the mechanical asymmetric response gel structure of the present invention (NR gel containing GO nanosheets having average lateral size=approximately 4 μm) within the strain range of −20 to +20% with right defined as positive.

FIG. 2B shows schematic of the buckling of GO nanosheets upon shear deformation. The arrow in the middle of the top row (⇔) is an indicator P (the longitudinal axis of the nanosheets) of GO nanosheets plane in the gel.

FIG. 2C Upper of FIG. 2C shows changes in the appearance of a 10 mm cube of the mechanical asymmetric response gel of the present invention upon shear deformation (+20% strain) with irradiating unpolarized light parallel to the y-axis. Lower of FIG. 2C shows relationship between the strain and light transmittance.

FIG. 2D Upper of FIG. 2D shows changes in the 2D (two-dimensional) SAXS image of a 1 mm-thick film gel structure of one example of the gel structure of the present invention upon shear deformation with irradiating X-ray parallel to the y-axis. Lower of FIG. 2D shows azimuthal angle-intensity plot of the 2D SAXS image.

FIG. 2E shows SEM images of GO sheets fixed in the silica network including cross-section SEM images of one example of the gel fixed by the silica network at the (i) left-sheared, (ii) original, and (iii) right-sheared states. Upper shows GO nanosheets embedded in the silica network, where the edges of GO nanosheets appeared as bright lines that are highlighted with multiple arrows, while the silica network was observed as a porous skeleton of ~5 nm spheres. Lower shows images of GO nanosheets exposing their surfaces (smooth, as highlighted by multiple arrows) at the cracks of silica network (porous), which were formed during cross-section cutting.

FIG. 3A shows vibration experiment of a 2 mm-thick gel film of one example of the mechanical asymmetric response gel structure of the present invention. (i) Schematic of the setup. (ii) Time course changes in the x-position of the bottom stage (input of vibration: input) and that of the top plate (output of vibration: output). (iii) Time course changes in the shear deformation of the gel estimated from (ii).

FIG. 3B shows vibration experiment of a 2 mm-thick gel film containing randomly oriented GO nanosheets as a comparative example of the present invention. (i) Schematic of the setup. (ii) Time course changes in the x-position of the bottom stage (input of vibration: input) and that of the top plate (output of vibration: output). (iii) Time course changes in the shear deformation of the gel film estimated from (ii).

FIG. 3C shows conversion of symmetric vibration applied from the bottom of the 2 mm-thick gel film of the one example of the mechanical asymmetric response gel structure of the present invention into asymmetric transport movement of water droplets on the surface. (i) Schematic of the setup. (ii) Time course pictures of asymmetric transport movement of water droplets.

FIG. 3D shows conversion of symmetric vibration applied from the bottom of the 2 mm-thick gel film of the one example of the mechanical asymmetric response gel structure of the present invention into asymmetric anti-gravity transport movement of water droplets on the surface. (i) Schematic of the setup. (ii) Time course pictures of asymmetric anti-gravity transport movement of water droplets.

FIG. 3E shows conversion of symmetric vibration applied from the bottom of a gel disk (gel disk made by combining multiple 2 mm-thick gel films) of one example of the mechanical asymmetric response gel structure of the present invention into directional rotation. (i) Schematic of the setup. (ii) Time course pictures of directional rotation.

FIG. 4A shows a schematic illustration showing asymmetric deformation of a 10 mm-thick gel slab of one example of the mechanical asymmetric response gel structure of the present invention upon local compression upon vertical indentation of a φ 2 mm cylinder laid parallel to the y-axis. (i and ii) Side-view pictures. (i) symmetric deformation of a gel slab with the same configuration containing randomly oriented GO nanosheets as a comparative example. (ii) asymmetric deformation of the gel slab of the one example of the present invention. (iii) Theoretical prediction of the deformation and strain distribution in the gel slab of the one example of the present invention based on the finite element analysis. In the figure, the arrow ⇔ is the indicator P in the nanosheets (the same as the longitudinal axis of the nanosheets).

FIG. 4B shows Time-lapse photo showing the trajectory of a q 2 mm metal ball free-dropped and bounced on a 2 mm-thick gel slab of one example of the mechanical asymmetric response gel structure of the present invention. In the figure, the arrow ⇔ is the indicator P in the nanosheets (the same as the longitudinal axis of the nanosheets).

FIG. 4C shows POM images of a gel in a 2 mm-thick gel slab containing titanium oxide nanosheets of one example of the mechanical asymmetric response gel structure of the present invention upon vertical indentation of a φ 50 μm microbead. To visualize deformed regions, the polarizer (depicted as P) and analyzer (depicted as A) were set parallel and perpendicular, respectively, to the x-axis. In the figure, the arrow ⇔×is the indicator P in the nanosheets (the same as the longitudinal axis of the nanosheets).

FIG. 4D shows directional migration of C. elegans on a 2 mm-thick gel slab of one example of the mechanical asymmetric response gel structure of the present invention. (i) POM images of a worm crawling on the surface of the gel slab (To visualize deformed regions, the polarizer (depicted as P) and analyzer (depicted as A) were set parallel and perpendicular, respectively, to the x-axis). (ii) Net movement (arrows) of individual worms crawling on a surface of a gel slab with the same configuration containing randomly oriented GO nanosheets as a comparative example. (iii) Net movement (arrows) of the worm on the surface of the gel slab of the one example of the mechanical asymmetric response gel structure of the present invention. (iv) Mean migration index of worms on the surface of the gel slab of each of the comparative example and the example of the present invention. Migration index was calculated as [(run distance to right)−(run distance to left)]/(total run distance). Each dot represents the migration index of a biologically independent assay comprised of at least 30 worms. Error bar indicates SEM. *** indicates p<0.001 (one-sided Mann-Whitney U test).

FIG. 5 shows shear test of a gel structure (R gel) containing randomly oriented GO nanosheets as a comparative example. (A) Schematic and optical pictures of a 10 mm cube of the R gel with randomly oriented GO nanosheets deformed in response to shear forces applied at the top of the gel. (B) Relationship between the displacement and applied force, which depicts the nonlinear and reciprocal feature of the R gel.

FIG. 6 shows a schematic illustration showing one example of the method for producing the mechanical asymmetric response gel structure of the present invention.

FIG. 7 shows a schematic illustration showing definitions of the nanosheet director (N), plane indicator (P), and tilt angle (θ) in the mechanical asymmetric response gel structure (NR gel) of the present invention. A x-y-z orthonormal coordinate is introduced, where the z-axis is parallel to gravity.

FIG. 8 shows asymmetric response of a NR gel to shear forces applied at a single point in 10 mm cubes of a NR gel of one example of the present invention. (A-C) the 10 mm cubes of the NR gel were sheared with applying shear forces at (A) the left end, (B) middle, and (C) right end of the upper face of the NR gel. (i) Optical images. (ii) Relationship between the displacement and applied force. In the figure, the arrow⇔(diagonal arrow) is the plane indicator P (the longitudinal axis) of the nanosheets in the NR gel.

FIG. 9 shows durability of a gel structure containing GO nanosheets (NR gel) of one example of the present invention upon repeated shear force sweep between −8 and +8 kPa over 100 cycles. (A) Changes in the stress-strain curve. (B) Changes in the 2D SAXS image. In the figure, the arrow⇔(diagonal arrow) is the plane indicator P (the longitudinal axis) of the nanosheets in the NR gel.

FIG. 10 shows cross-section SEM images of a NR gel fixed at the left-sheared state in the FIG. 2B (the left illustration in the FIG. 2B). (i) GO nanosheets embedded in silica network, where the edges of GO nanosheets appeared as bright lines, while the silica network was observed as a porous skeleton of ~5 nm spheres. (ii and iii) GO nanosheets exposing their surfaces at the cracks of silica network, which were formed during cross-section cutting.

FIG. 11 shows cross-section SEM images of a NR gel fixed at the original state in which no shear force is applied in the FIG. 2B (the middle illustration in the FIG. 2B). (i) GO nanosheets embedded in silica network, where the edges of GO nanosheets appeared as bright lines, while the silica network was observed as a porous skeleton of ~5 nm spheres. (ii and iii) GO nanosheets exposing their surfaces at the cracks of silica network, which were formed during cross-section cutting.

FIG. 12 shows cross-section SEM images of a NR gel fixed at the right-sheared state in the FIG. 2B (the right illustration in the FIG. 2B). (i) GO nanosheets embedded in silica network, where the edges of GO nanosheets appeared as bright lines, while the silica network was observed as a porous skeleton of ~5 nm spheres. (ii and iii) GO nanosheets exposing their surfaces at the cracks of silica network, which were formed during cross-section cutting.

FIG. 13 shows effect of tilt angle θ on a degree of non-reciprocity of a NR gel (E_{R}/E_{L}) of one example of the gel structure of the present invention. In the figure, the arrow⇔(diagonal arrow) is the plane indicator (the longitudinal axis) P of the nanosheets in the NR gel. (i) Stress-strain curves of the samples of the NR gel, for each of which the elastic moduli at −20% (E_{L}) and +20% (E_{R}) strains were estimated as the average tangent. (ii) Changes in the degree of non-reciprocity depending on the tilt angle θ.

FIG. 14 shows shear response of a R gel (containing GO sheets) with a symmetric structure as a comparative example. (A and B) stress-strain curve of each of the gels with the tilt angles θ of (A) 0° and (B) 90°. In the figure, the arrow (⇔) is the plane indicator (the longitudinal axis) P of the nanosheets in the gel.

FIG. 15 shows effect of size of GO nanosheets on a degree of non-reciprocity of a NR gel (E_{R}/E_{L}) of one example of the gel structure of the present invention. In the figure, the arrow⇔(diagonal arrow) is the plane indicator (the longitudinal axis) P of the nanosheets in the NR gel. (A) TEM images (upper) and size distributions (lower) of (i) the GO nanosheets (average lateral size (average size of the short axis)=~4 μm) and (ii) the chopped GO nanosheets (average lateral size (average size of the short axis)=~0.5 μm). (B) Stress-strain curve from −40% to +40% strain of the NR gel comprising (ii) the chopped GO nanosheets.

FIG. 16 shows examples of NR gels of the present invention with various concentrations of GO nanofillers. (i) Stress-strain curves of the samples of the NR gel, for each of which the elastic moduli at −20% (E_{L}) and +20% (E_{R}) strains were estimated as the average tangent. (ii) Changes in the degree of non-reciprocity depending on the concentration of the GO nanosheet. In the figure, the arrow⇔(diagonal arrow) is the plane indicator (the longitudinal axis) P of the nanosheets in the NR gel.

FIG. 17 shows graph showing degrees of non-reciprocity (E_{R}/E_{L}) of examples of NR gels of the present invention with various nanofillers materials. Stress-strain curves at −20% (E_{L}) and +20% (E_{R}) strains were obtained, and the respective elastic moduli were estimated from the average tangent. In the figure, the arrow⇔(diagonal arrow) is the plane indicator (the longitudinal axis) P of the nanosheets in the NR gel.

FIG. 18 shows samples of NR gels of the present invention in which the types of polymers having a crosslinked structure that constitute the matrix are different from each other. (i) Stress-strain curves of the samples of the NR gel, for each of which the elastic moduli at −20% (E_{L}) and +20% (E_{R}) strains were estimated as the average tangent. (ii) a value of the degree of non-reciprocity (E_{R}/E_{L}) of each of the samples of the NR gel. In the figure, the arrow⇔(diagonal arrow) is the plane indicator (the longitudinal axis) P of the nanosheets in the NR gel.

FIG. 19 shows more detailed results of directional transport of water droplets caused by the asymmetric vibration from the NR gel film of the one example of the present invention shown in FIG. 3C. Time course changes in the x-position of the water droplets in the experiment of FIG. 3C. In the figure, the arrow⇔(diagonal arrow) is the plane indicator (the longitudinal axis) P of the nanosheets in the NR gel.

FIG. 20 shows more detailed results of directional rotation of a wheel caused by the asymmetric vibration from the disk of the NR gel of the one example of the present invention shown in FIG. 3E. (A) Left is schematic of the disk of the NR gel, and Right is POM image with a retardation plate of the gel disk. (B) Time course changes in the rotation angle of the wheel in the experiment of FIG. 3E. In the figure, the arrow⇔(diagonal arrow) is the plane indicator (the longitudinal axis) P of the nanosheets in the NR gel.

FIG. 21 shows visualization of the buckling of GO nanosheets in a 10 mm-thick NR gel as one example of the present invention upon local compression. (A) Schematic of the configuration of the NR gel slab, indentation direction, incident light direction, and observation direction. (B) Changes in the appearance of the NR gel slab before and after the local compression. In the figure, the arrow⇔(diagonal arrow) is the plane indicator (the longitudinal axis) P of the nanosheets in the NR gel.

FIG. 22 shows POM images of C. elegans crawling on a surface of a NR gel film of one example of the present invention.

FIG. 23 shows responses of the fish bone-shaped frameworks composed of a R gel of a comparative example and a NR gel of one example of the present invention to shear forces. (A) shows the R-gel, and (B) shows the NR-gel, showing the results of shearing the center plate by applying forces at the center plate toward left and right. (i) Optical images. (ii) Relationship between the displacement and applied force. In the figure, the arrow⇔(diagonal arrow) is the plane indicator (the longitudinal axis) P of the nanosheets in the NR gel.

FIG. 24 shows one example of a setup for preparing a NR gel of one example of the present invention. (A) Schematic for the whole setup. (B) Schematic for the arrangement of the rotation stage, spacer, cuvette filled with pre-gel, cooling box, and rotation axis of the stage.

FIG. 25 shows a setup for shear deformation experiments. (A) Schematic for the configuration of the clamps, zigs, and hydrogel sample. (B) Optical image of the setup.

FIG. 26 shows a setup for applying vibration. (A) Schematic for the arrangement of the waveform generator, the electrodynamic shaker, and the sample holder. The arrows in (A) indicate the direction of vibration. (B) Optical image of the whole setup.

FIG. 27 shows one example of mechanism for a buckling of GO nanosheets and an emergence of mechanical non-reciprocity in a NR gel of one example of the present invention. (A and B) Schematic representation of the NR gel upon shearing to the left (A) and right (B). (i) Decomposition of the shear force into the compression and tension forces. In the figure, the arrow⇔(diagonal arrow) is the plane indicator (the longitudinal axis) P of the nanosheets in the NR gel. (ii) Conceptual scheme of inner structure of the NR gel for expressing the states of a GO nanosheet, polymer chains in the matrix, and anchoring points between a GO nanosheet and polymer chains.

FIG. 28 shows the x₁-x₂-x₃ orthonormal coordinate prescribed as the material's principal directions with the x₁-x₂ plane parallel to the nanofillers in the NR gel.

FIG. 29 shows Young's moduli, E₁ and E₃., under uniaxial tension and compression in the x₁- and x₃-directions.

$${E}_{1}^{+} under {ε}_{11} \geq 0 , \qquad ( A )$$

$${E}_{1}^{-} under {ε}_{11} < 0 , \qquad ( B )$$

$${E}_{3}^{+} under {ε}_{33} \geq 0 , and \qquad ( C )$$

$${E}_{3}^{-} under {ε}_{33} < 0. \qquad ( D )$$

FIG. 30 shows Poisson's ratios, v₁₂, v₁₃, and v₃₁(=v₃₂), under uniaxial tension and compression in the x₁- and x₃-directions. (A) v₁₂ and v₁₃ under ε₁₁≥0, (B) v₁₂ and v₁₃ under ε₁₁<0, (C) v₃₁ under ε₃₃≥0, and (D) v₃₁ under ε₃₃<0.

FIG. 31 shows shear moduli,

$${\overline{G}}_{13} and {\overline{G}}_{31} ,$$

as a function of |du₁/dx₃| and |du₃/dx₁|.

$${\overline{G}}_{13} and \qquad ( A )$$

$${\overline{G}}_{31} . \qquad ( B )$$

FIG. 32 shows comparison of the experimental and theoretical responses of the shear stress-shear strain, σ_{zx} and 2ε_{zx}. (A) Relation between x-y-z and x₁-x₂-x₃ orthonormal coordinates. (B) Shear stress-shear strain curve.

FIG. 33 shows initial configuration (u=0 mm) and boundary conditions of the two-dimensional finite element model for cylindrical indentation analysis.

FIG. 34 shows a deformed configuration of a NR gel film of one example of the present invention at u=1 mm and strain distributions. (A) 2ε_{zx}, (B) ε₁₁ and (C) ε₃₃.

### DESCRIPTION OF EMBODIMENTS

The present invention relates to a gel structure showing asymmetric response to mechanical force. First, the asymmetric response to mechanical force will be described.

This feature can also be expressed as an asymmetric mechanical response. It can also be expressed as mechanical non-reciprocity, which transmits a mechanical quantity asymmetrically between two distant points. Such feature makes it possible to guide, damp and control mechanical energy. One example of the gel structure of the present invention shows more than 60 times higher elastic modulus when sheared one way than the opposite. Consequently, it can transform symmetric vibrations into asymmetric ones that are applicable for mass transport and energy harvest. Furthermore, the gel structure of the present invention exhibits asymmetric deformation when subjected to local interactions, and thus, can induce the directional motion of a wide range of objects (from macroscopic ones to small living creatures). The present invention can promote the development of non-reciprocal systems toward practical applications such as energy conversion and biological manipulation.

In many physical systems, the law of reciprocity ensures that signal propagation through the system always occurs in symmetric manner. In simpler words, if a system responds to an input in one way, it also does the same when the input is applied in the opposite direction. Breaking this symmetry induces non-reciprocity. The non-reciprocity is a feature that has been increasingly pursued over the past several years in various fields, including optics (References 1 to 4), acoustics (References 5 to 7), quantum systems (References 8, 9), and mechanics (References 10, 11). Systems exhibiting non-reciprocity to mechanical quantities have recently emerged due to their potential to guide, damp, and control mechanical signals and energies, in ways that have not been imagined for reciprocal systems (References 12 to 14). To realize such systems, breaking of either the time-reversal symmetry (dynamic systems) or the material deformation symmetry (static systems) is required. One simple example is the non-reciprocal transmission of the displacement field that is realized by processing silicon rubber into fishbone-structured metamaterial (References 10). However, mechanical non-reciprocity has so far mainly been realized through the complicated design of active robotics (References 15, 16) or metamaterial frameworks (References 17, 18). Even the most advanced systems still rely on the shaping and connection of reciprocal materials, which limits the design freedom and practical applications of such systems. The present invention is based on a technical concept that differs from these conventional techniques. The present inventors have realized mechanical non-reciprocity in a material as its intrinsic property, without requiring the material to have a complex shape or structure. Such material is expected to show non-reciprocal response in any condition regardless of shape and size, and in principle can manipulate any objects with which it interacts.

Next, the gel structure of the present invention will be described.

The present invention relates to a gel structure showing asymmetric response to mechanical force (sometimes referred to as “non-reciprocal gel (NR gel)” in the description and drawings). One example of the gel structure of the present invention is a gel structure comprising a gel composition including at least one type of anisotropic nanostructures, at least one type of liquid, and at least one type of polymer having a crosslinked structure.

There are no particular limitations on the at least one liquid in the present invention. The liquid may be a mixture of different types of liquids. It is preferably water. That is, one preferred example of the present invention is a hydrogel structure.

There are no particular limitations on the at least one type of anisotropic nanostructures in the present invention as long as they are nanostructures having shape anisotropy. Shape anisotropy refers to a shape having a longitudinal axis, and may be one-dimensional (linear), two-dimensional (sheet-shaped), or three-dimensional. For example, sheet-shaped nanostructures, so-called “nanosheets,” may be used as the anisotropic nanostructures. A nanosheet is a two-dimensional structure with a thickness of about one atom. Commercially available products or nanosheets prepared based on various reports (such as References 26, 27) can be used. Examples of nanosheets that can be used in the present invention include those described in JP Patent Publication (Kokai) No. 2021-95306 and JP Patent Publication (Kokai) No. 2018-200233. As the anisotropic nanostructures, those that are generally called “fillers” can be used, and fillers of various anisotropic shapes, such as one-dimensional shapes and two-dimensional shapes, can be selected depending on the average size of the longitudinal and short axes, depending on the purpose.

There are no particular limitations on materials for the anisotropic nanostructures. In the gel structure of the present invention, preferably, anisotropic nanostructures are oriented with their longitudinal axes aligned. In this respect, preferably the anisotropic nanostructures are oriented by a predetermined physical stimulus. That is, preferably, they are selected from materials that are sensitive to the physical stimuli used to orient them. For example, when orientation is achieved by applying a magnetic field, a metal oxide that is magnetically oriented is preferred. Examples of nanosheets include graphene oxide nanosheets, niobium oxide nanosheets, titanium oxide nanosheets, clay nanosheets, alumina nanotubes, and carbon nanotubes. In addition, the anisotropic nanostructures are not necessarily in a solid form. For example, the anisotropic nanostructures may be nanochannel pores that are formed after removing anisotropic nanostructures consisting of a solid.

There are no particular limitations on the content of anisotropic nanostructures. For example, the content may be 0.005 to 5 wt %, 0.01 to 1 wt %, or 0.05 to 0.5 wt %.

The at least one type of polymer having a crosslinked structure in the present invention may be any of an organic polymer, an inorganic polymer, or an organic-inorganic hybrid polymer. The polymer having a crosslinked structure can be obtained by polymerizing a polymerizable composition. The polymerizable composition is a composition including at least one type of polymerizable monomer and at least one type of polymerization initiator. It may also include at least one type of crosslinking agent. Examples thereof include polyacrylamides having a cross-linked structure and polysiloxanes having a cross-linked structure.

One example of the gel structure of the present invention is a gel structure which has at least one surface in a stationary state, and in which longitudinal axes of the at least one type of anisotropic nanostructures are oriented approximately parallel to each other and non-parallel and non-perpendicular to the at least one surface. “Non-parallel and non-perpendicular” means greater than 0° and less than 90° One example is 15° to 75°, another example is 20° to 70°, and another example is 30° to 60°.

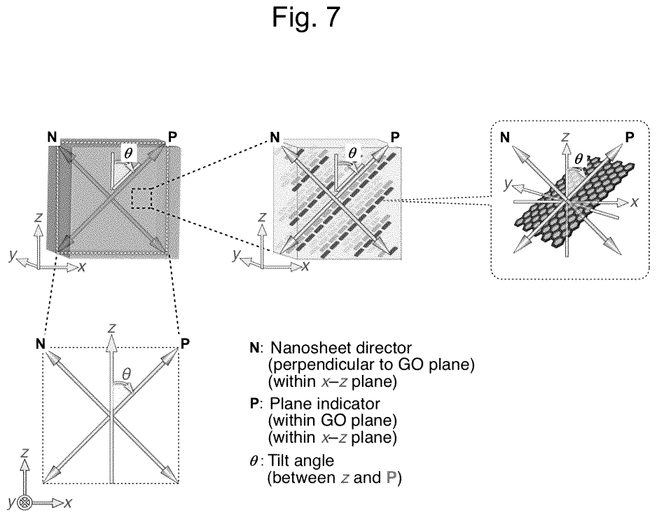

FIG. 7 is a schematic illustration showing the definition of the GO nanosheet director (N), GO nanosheet plane indicator (P), and tilt angle (θ) for the gel structure (NR gel) containing GO nanosheets as an example. In this example, the plane indicator (P) is the longitudinal axis. In the figure, a x-y-z orthonormal coordinate is introduced, where the z-axis is parallel to gravity. GO nanosheets in the NR gel adopt a unidirectional alignment; in other words, their longitudinal axes are oriented approximately parallel to each other (i.e., all nanosheets orient their normal vector in one direction). Furthermore, the GO sheets in the NR gel are oriented with their longitudinal axes at the tilt angle θ (0°<θ<90°) relative to the top (and bottom).

In the gel structure of the present invention, the anisotropic nanostructures are embedded in a polymer matrix having a crosslinked structure and is fixed in the above-mentioned state.

There are no particular limitations on the structure of the gel structure of the present invention, and the structure may have a shape suitable for the application. Examples thereof include film shape and slab shape. Furthermore, a plurality of gel structures of the present invention can be integrated and used in combination. In this example, the tilt angles of the longitudinal axes of the anisotropic nanostructures contained in the plurality of gel structures used may be the same or different from each other.

There are no particular limitations on the method for producing a gel structure of the present invention. One example is a method for producing a gel structure, comprising: a first step of accommodating a gel raw material composition, which includes at least one type of anisotropic nanostructures, at least one type of liquid, and a polymerizable composition, in a container having at least one identifiable face; and a second step of carrying out a polymerization reaction of the polymerizable composition in the container, wherein in the second step, the at least one type of anisotropic nanostructures is oriented with their longitudinal axes approximately parallel to each other and non-parallel and non-perpendicular to the at least one identifiable face. In the second step, a physical stimulus may be applied to the gel raw material composition to orient the anisotropic nanostructures in a desired orientation pattern. The physical stimulus applied to the gel raw material composition is a stimulus that can control the orientation of the anisotropic nanostructures, and can be selected depending on the properties, shape, or the like of the anisotropic nanostructures and the desired orientation pattern. Examples of physical stimuli include a magnetic field, an electric field, a mechanical force, a concentration gradient, and a temperature gradient, and two or more stimuli selected from these may be combined. The two or more physical stimuli may be applied simultaneously or at different times.

One example is a method comprising: a first step of accommodating a gel raw material composition including at least one type of anisotropic nanostructures, at least one type of liquid, and a polymerizable composition in a container that can rotate about an arbitrary axis; a second step of carrying out a polymerization reaction of the polymerizable composition in the container, wherein in the second step, the container is rotated about the arbitrary axis, if necessary, depending on the orientation pattern of the anisotropic nanostructures, during which a physical stimulus is applied in a direction that is non-parallel and non-perpendicular to the arbitrary axis.

In the case of using a material whose longitudinal axis is oriented parallel to a magnetic field, such as graphene oxide (GO) nanosheets, as anisotropic nanostructures, it is preferable to control the orientation in the second step by applying a magnetic field as a physical stimulus, as well as other physical stimuli (such as the concentration gradient and temperature gradient). In the case of using a material whose longitudinal axis is oriented perpendicular to a magnetic field, such as titanium oxide nanosheets, as anisotropic nanostructures, the orientation can be controlled by applying only a magnetic field as a physical stimulus.

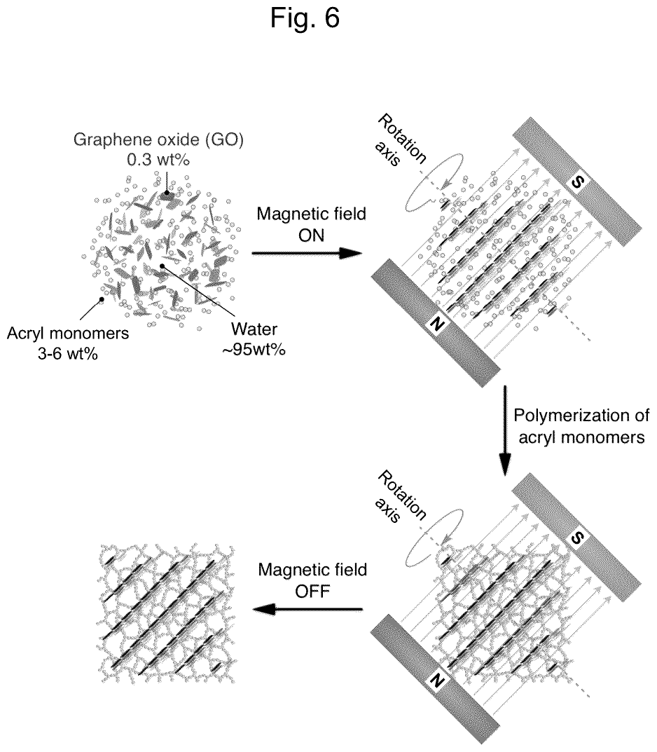

FIG. 6 is a schematic illustration showing the outline of the step of preparing a gel structure with unidirectionally aligned GO nanosheets as one example of the above-described method. In this example, a pre-gel solution including polyacrylamide (e.g., acrylamide as a monomer and bisacrylamide as a crosslinking agent) as a polymer having a crosslinked structure, GO nanosheets, and water was prepared. The pre-gel solution poured in a cuboid-shaped cuvette was cooled to approximately 0° C. and subjected to a 10 T rotating magnetic field in a tilted geometry until when the unidirectional alignment of GO nanosheets was achieved. The pre-gel solution was gradually warmed to 25° C., so that the free radical polymerization proceeded to afford a cuboid of the NR gel. After polymerization, GO nanosheets were fixed by the polymer network, so that their orientation order was not lost even though the magnetic field was turned off.

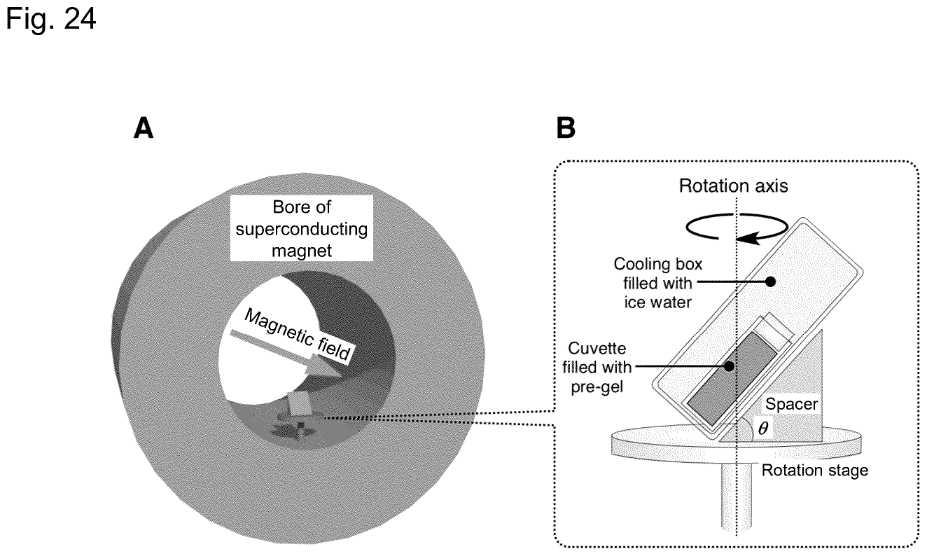

FIG. 24 is a schematic illustration showing one example of the setup used for the above-described production method: (A) a schematic illustration of the whole setup; (B) a schematic illustration of the arrangement of the rotation stage, spacer, cuvette filled with pre-gel, cooling box, and rotation axis of the stage.

In one example of the gel structure of the present invention, when the bottom face is fixed to a floor and a left or right shear force is applied to the top face, the difference in elastic modulus between both is 10 times or more. Another example of the gel structure of the present invention is a gel structure that has at least one surface in a stationary state, and that exhibits elastic moduli of different magnitudes when shear forces of the same magnitude are applied in a first direction and a second direction different from the first direction in a plane parallel to the one surface. The mechanical reciprocity shown in this example can be readily demonstrated by the following experiment.

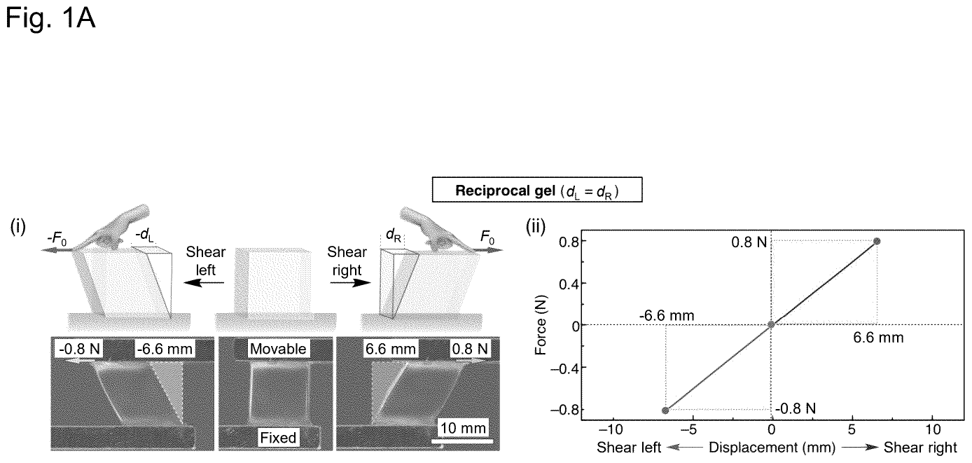

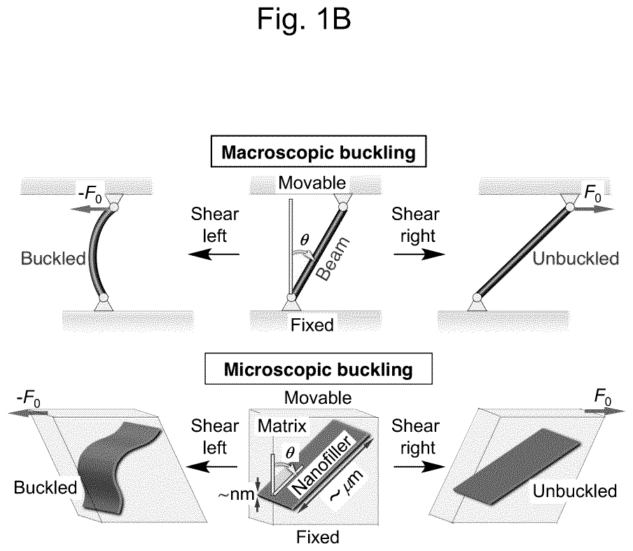

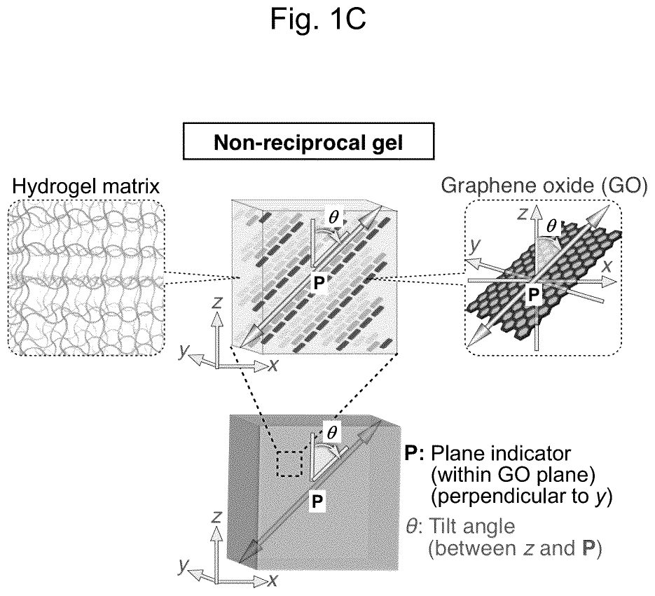

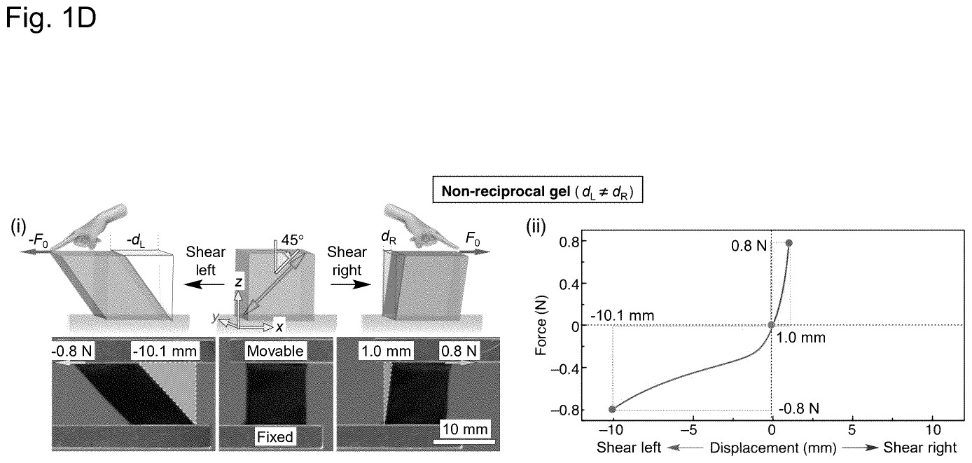

As an example, a hydrogel structure consisting of a polyacrylamide network as the matrix and graphene oxide (GO) nanosheets as the nanofillers is prepared. In this gel, the nanosheets are unidirectionally oriented with their longitudinal axes (sheet planes) in a tilted manner as shown in FIG. 1C, just like the tilted beam in FIG. 1B. GO nanosheets easily buckle when subjected to in-plane compression owing to their single-atom thickness (References 21, 22). A plane indicator P (longitudinal axis) is defined to help understand the orientation of GO plane, and the angle between the height direction and P is denoted as the tilt angle θ (details in FIG. 7). FIG. 1D shows the deformation of a 10 mm cube of such composite hydrogel with θ=45° when it was sheared to the right (left) with an applied force F₀=0.8N (−F₀=−0.8N). Surprisingly, the hydrogel exhibited an almost 10 times larger displacement when it was sheared to the left than to the right. The force-displacement relationship also depicted the highly asymmetric response of the hydrogel, which is a strong evidence of mechanical non-reciprocity (FIG. 1D, ii).

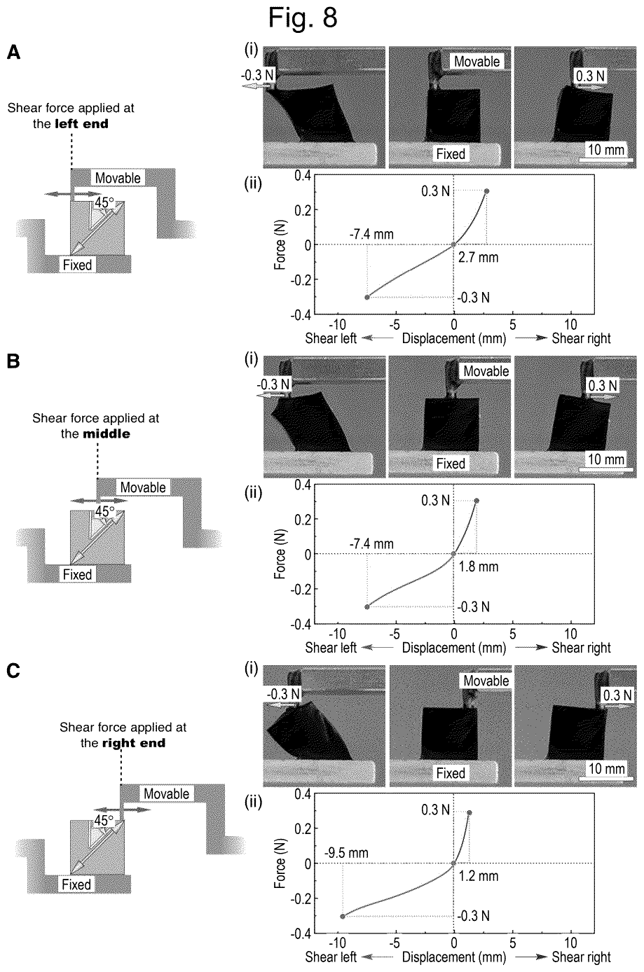

The present inventors also confirmed that the hydrogel showed a similar asymmetric behavior even when the shear force was applied at a single point (FIG. 8). The hydrogel is denoted as the non-reciprocal (NR) gel, and the directions exhibiting larger and smaller shear deformation are denoted as the softer and harder directions, respectively.

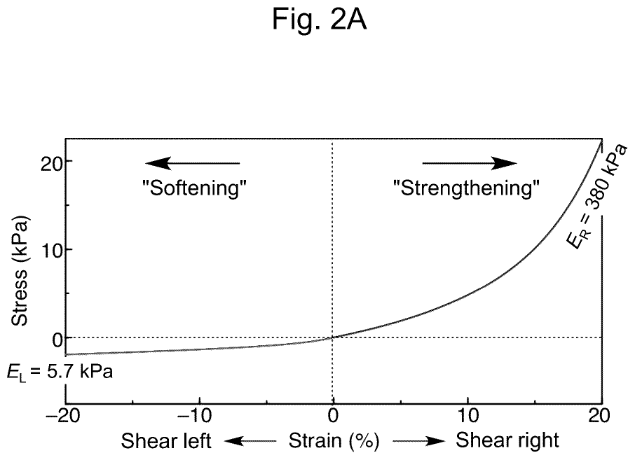

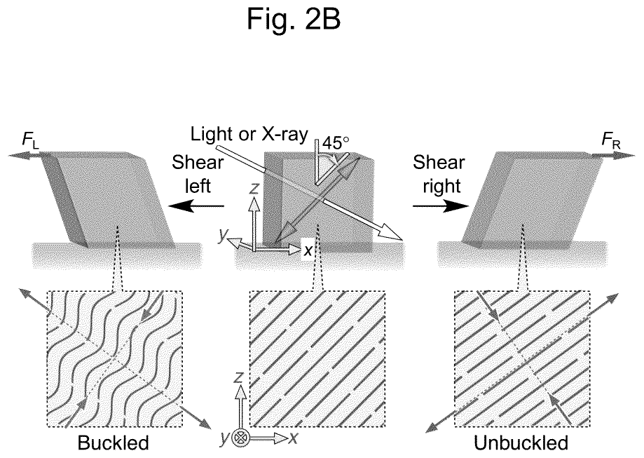

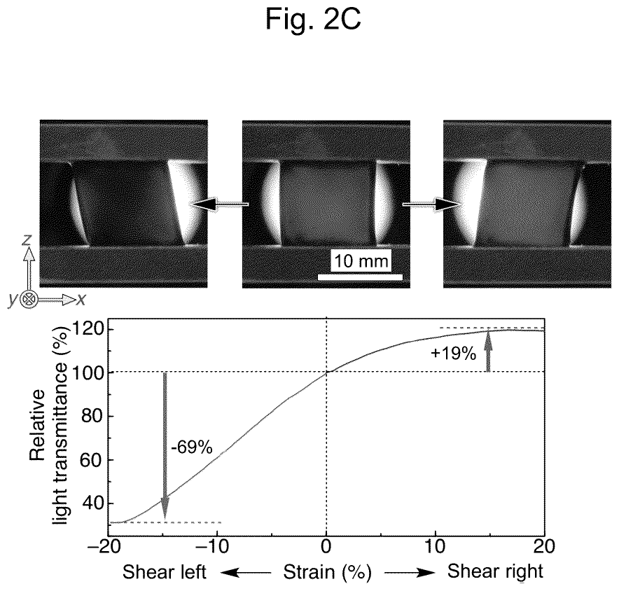

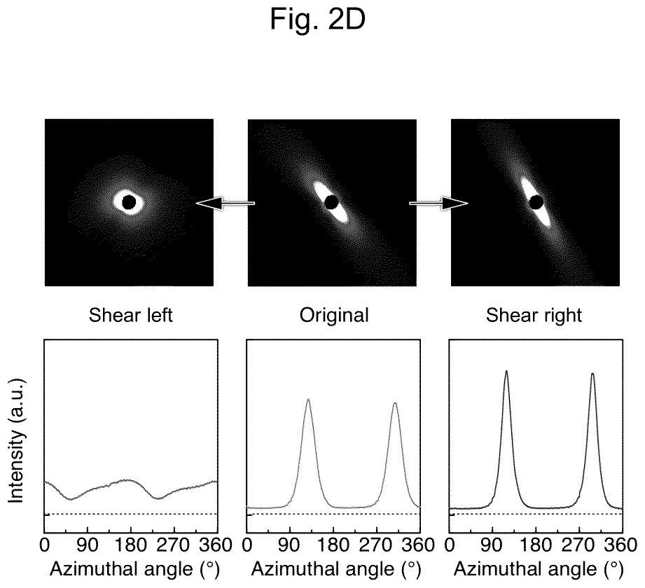

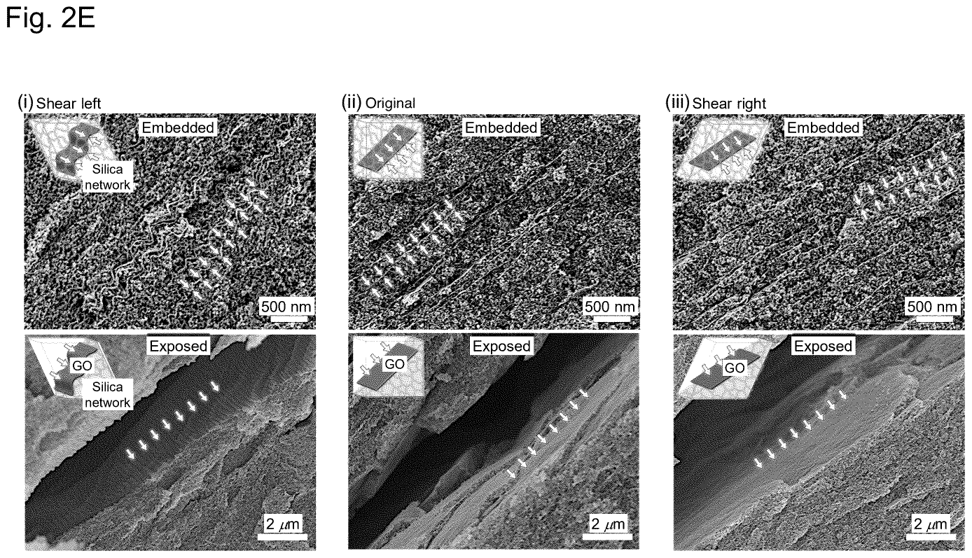

For more quantitative analysis of the asymmetric response to shear, the stress-strain relationship of the NR gel was measured within the strain range of ±20% (FIG. 2A). Upon changing the left-sided strain up to −20%, the elastic modulus continuously decreased, resulting in a sluggish change in the stress (FIG. 2A). In contrast, upon changing the right-sided strain up to +20%, the elastic modulus was significantly enhanced with an increase in the strain, so that the strain-stress relationship exhibited a typical J-shaped curve (FIG. 2A). An extremely large difference existed between the elastic modulus at the left-shear strain of −20% (E_{L}) and at the right-shear strain of +20% (E_{R}); E_{R} and E_{L} were estimated to be 380 kPa and 5.7 kPa, respectively.

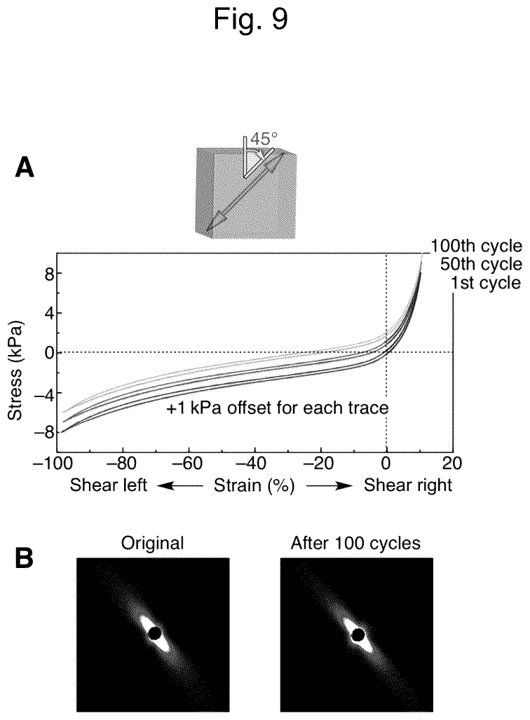

To confirm whether this asymmetric response really originates from the direction-dependent buckling of nanofillers (FIG. 2B), the present inventors monitored the changes in the alignment of GO nanosheets during the shear deformation by investigating the light transmittance of NR gel along the y-axis (FIG. 2C and movie S2). Light transmittance is an effective indicator of the GO orientation since its light-absorbing efficiency highly depends on the angle between GO plane and light path. The original NR gel showed a considerable light transmittance since all GO nanosheets exposed their edges to the light source (FIG. 2C, mid). Upon shearing to the left (softer) up to −20% strain, the light transmittance dropped in 69% (FIG. 2C, left), indicative of the disordering of the GO orientation due to buckling. In contrast, upon shearing to the right (harder) up to +20% strain, the light transmittance increased (FIG. 2C, right). This was because the alignment degree of GO nanosheets was enhanced since they experienced a tensile force along the same direction they were aligned (shearing-induced alignment). Another proof for the direction-dependent buckling is the small-angle X-ray scattering (SAXS) analysis, which is often used to evaluate the ordering of nanostructures. When an X-ray beam was passed through to the NR gel along the y-axis, a highly anisotropic scattering pattern was observed, which proved the high ordering of GO nanosheets in the original NR gel (FIG. 2D, mid). Upon shearing to the left (softer), the scattering pattern became isotropic (FIG. 2D, left), indicating the complete disordering of the GO orientation. In contrast, upon shearing to the right (harder), the anisotropic scattering pattern became sharper (FIG. 2D, right), which was consistent with the result of light transmittance measurement (FIG. 2C, right). The transition between the ordered and disordered states was reversible and durable, as demonstrated by a cycling test of over 100 cycles (FIG. 9). The cycling test also revealed that the stress-strain curve showed little hysteresis (FIG. 9A), indicating that the NR gel was highly elastic, favorable for transferring mechanical energies in an efficient manner. Even after 100 cycles of shear force sweep, the highly asymmetric response to shear and the highly oriented structure of the GO nanosheets showed little deterioration, indicating excellent reversibility and durability of the direction-dependent buckling of the GO nanosheets in the NR gel.

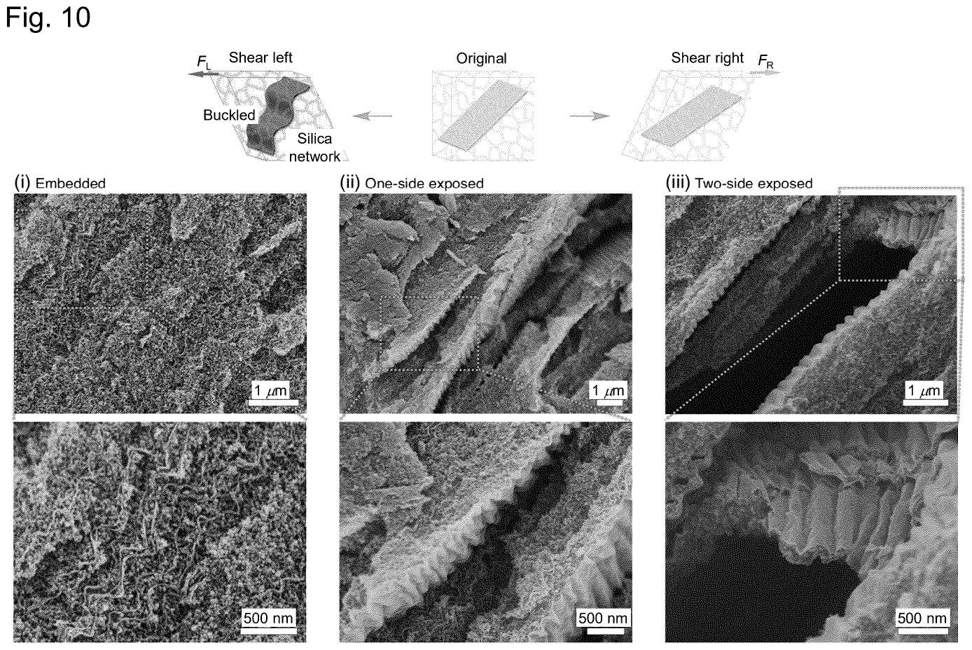

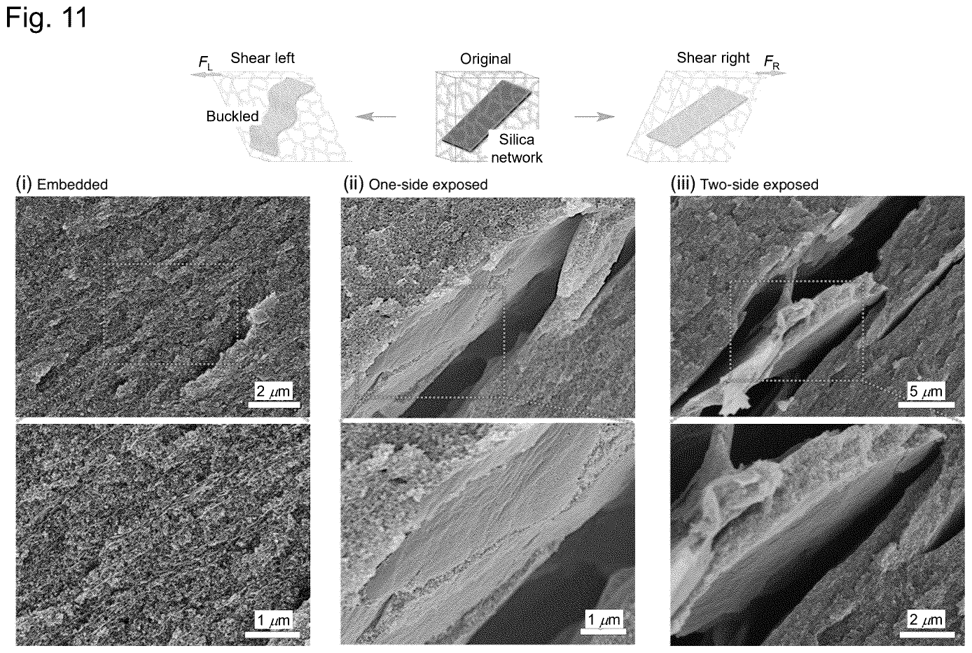

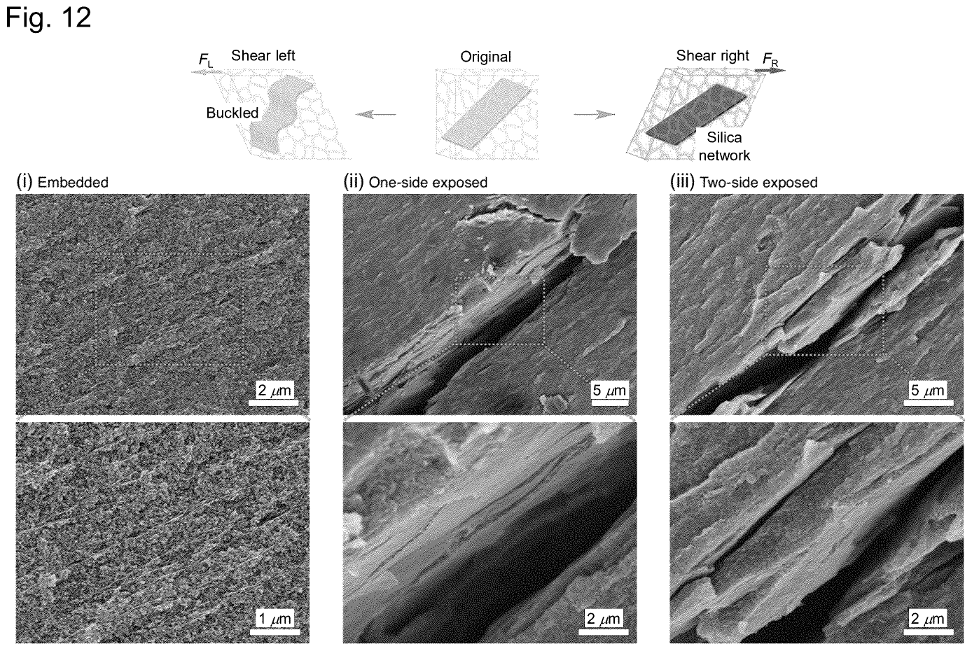

By using scanning electron microscopy (SEM), the present inventors succeeded in visualizing the buckling of GO nanosheets in a more direct manner. For SEM analysis, samples of the NR gel in the original, left-sheared, and right-sheared states were rigidified by a silica network, so that the configuration of GO nanosheets at each state was fixed (details in the Examples). In the cross-section images of the left-sheared (softer) sample (FIG. 10), all GO nanosheets were buckled with a regular periodicity of approximately 300 nm along the direction they were compressed. Such buckling could be more easily visualized when the nanosheet surface was exposed due to cracks during cross-section cutting. In contrast, the SEM images of the original-state sample (FIG. 11) and the right-sheared (harder) sample (FIG. 12) did not show any sign of such periodic buckling. For fair comparison between these three states, SEM images taken under the similar magnifications and observation directions were summarized in FIG. 2E. These images verified that the buckling of GO nanosheets is a shear direction-dependent phenomenon. Such direction-dependent behavior causes a large difference in the reinforcement ability of GO nanosheets under the left and right shear, thus giving birth to the non-reciprocity (details in 2.1 in the Examples).

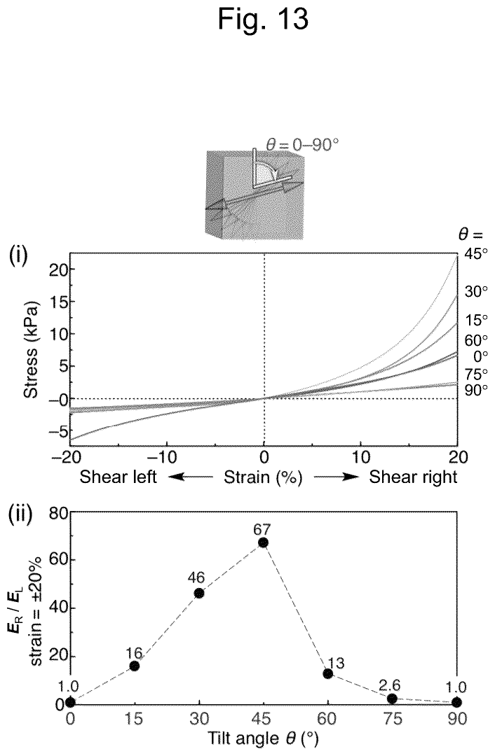

Then, various NR gels with different tilt angles θ of the GO nanosheets were prepared, and the influence of the tilt angle θ of the longitudinal axis of the GO sheets was investigated. The results are shown in FIG. 13. From the results, it can be understood that in the gel structure of the present invention, when the tilt angle of the longitudinal axis of the nanosheets relative to the top or bottom face is greater than 0° and less than 90°, the gel structure exhibits asymmetric responsiveness to mechanical force (e.g., shear force). In this example, it can also be understood that there is a tendency that upon increasing θ from 0° to 90°, E_{R}/E_{L} increases, reaching the maximum at θ=45°, and then decreases.

The asymmetric shape of the plot in the graph of FIG. 13 (ii) can be explained with taking account of the following two points.

First, although the degree of non-reciprocity is the ratio between the elastic moduli under the right shear (harder) and the left shear (softer), it is indeed determined mainly by the behavior of GO under the right shear (harder). Under the left shear (softer), the buckling of GO nanosheets occurs easily irrespective of tθ (0°<θ<90°), where the reinforcement effect of GO nanosheets disappears since the mechanical performance of the gel is no longer related with the arrangement of GO. Indeed, the stress-strain curves in FIG. 13 (i) indicate that all the gels (0°<θ<90°) exhibit similar curves when sheared to left (softer). Meanwhile, when sheared to the right (harder), GO nanosheets continue working as reinforcers, where their performance is critically influenced by θ. Overall, the degree of non-reciprocity is determined by how GO nanosheets efficiently enhance their working under the right shear (harder).

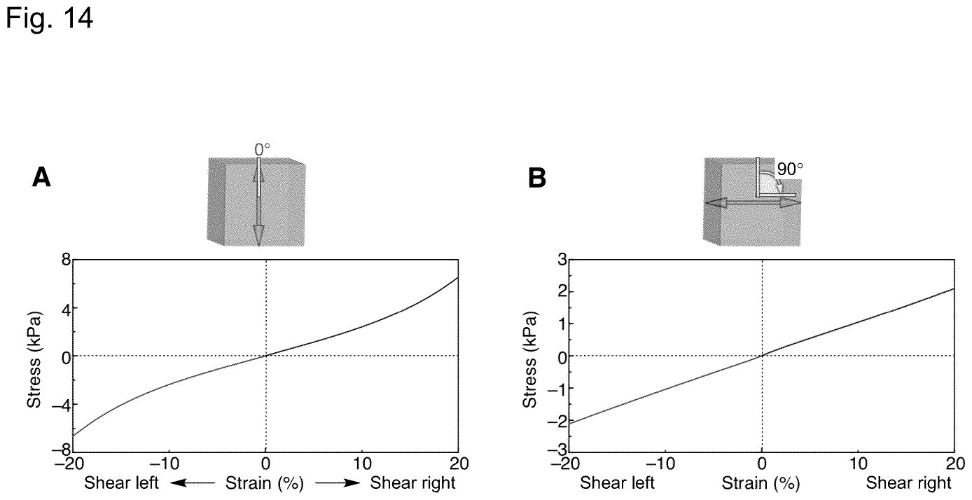

Second, since the structures of the gels at θ=0° and θ=90° are both symmetric, the shear response curves are completely symmetric. When the tilt angle θ=0° in the reciprocal gel (R gel) shown in FIG. 14 (A), the shear toward both sides leads to the stretch of GO nanosheets, so that two symmetric J-curves can be observed. However, the R gel with the tilt angle θ=90° shown in FIG. 14 (B) is in a quite specific state at which GO nanosheets has no contribution as reinforcers under shear deformation. Indeed, in the shear test of the gel with θ=90°, it can be seen that the stress increases only sluggishly and linearly upon increasing the strain, unlike the case of θ=0°.

Taking into account these two points, it is reasonable that the degree of non-reciprocity abruptly decreases when θ becomes close to 90° in the NR gel of the present invention, thereby elucidating the asymmetric shape of the plot in the graph of FIG. 13 (ii).

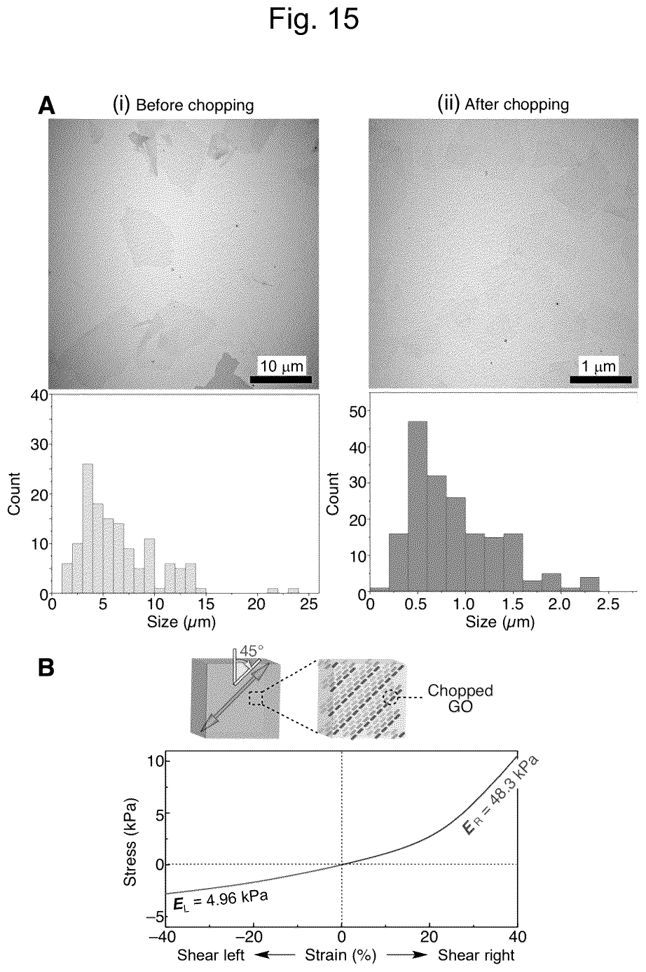

In addition, two samples were prepared as examples of the gel structure of the present invention, which were prepared under the same conditions except that GO nanosheets of different sizes were used, and the effect of the size of the short (lateral) axis (lateral size) of the nanosheets was investigated. The results are shown in FIGS. 2A and 15B. FIG. 2A shows a stress-strain curve of a NR gel containing GO nanosheets with an average size of the short axis (average lateral size) of approximately 4 μm shown in FIG. 15A. FIG. 15B shows a stress-strain curve of a NR gel containing GO nanosheets of smaller sizes with an average size of the short axis (average lateral size) of approximately 0.5 μm shown in FIG. 15A. It can be understood that both exhibits asymmetric responsiveness to mechanical force (e.g., shear force). The comparison of FIGS. 2A and 15B indicates that the larger GO nanosheets (FIG. 2A) can realize a higher degree of non-reciprocity due to the following reason.

Under the left (softer) shear, the elastic modulus was hardly influenced by the GO size, because this shear induced the buckling of GO nanosheets and deprived their reinforcing ability, irrespective of their sizes. Meanwhile, under the right (harder) shear, the elastic modulus was enhanced as the nanofillers became larger, because GO nanosheets were hardly buckled in this shear and exhibited their intrinsic performance. In this case, it can be said that larger nanofillers serve as better reinforcers. That is, it can be said that the anisotropic nanostructures used in the present invention have a more pronounced asymmetric responsiveness when they have a two-dimensional sheet shape than when they have a one-dimensional shape.

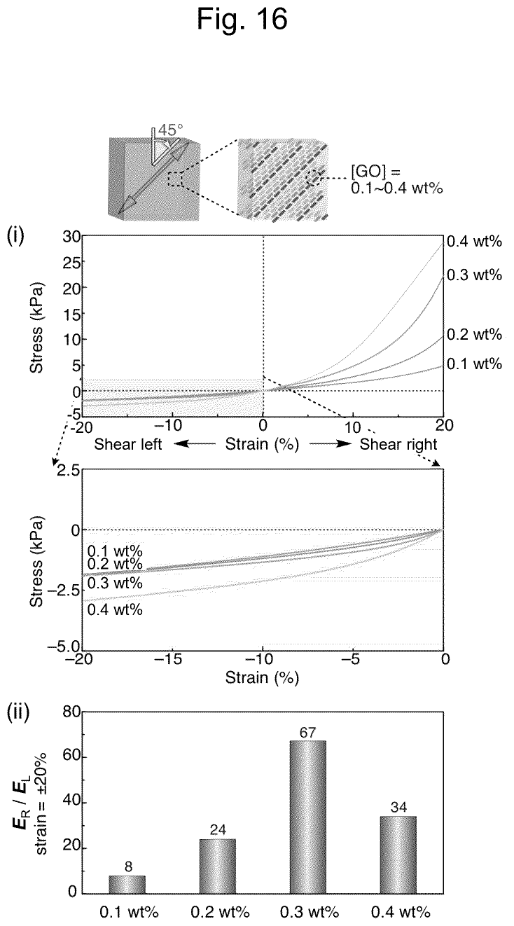

In addition, various samples serving as examples of the gel structure of the present invention were prepared under the same conditions except that GO nanosheets had different concentrations, and the effect of concentration was examined. The results are shown in FIG. 16. The results shown in FIG. 16 indicate that non-reciprocity can be obtained even at low concentrations of GO nanosheets (0.1 wt %). The effect of the concentrations of GO nanosheets shows that, as the concentration of GO increases, E_{R}/E_{L} tends to be higher. In this example, up to the, E_{R}/E_{L} reached a maximum value at 0.3 wt % and then decreased. The reason for this is presumed to be as follows.

Under the left (softer) shear, the elastic modulus was hardly influenced by the GO concentration due to buckling. Meanwhile, under the right (harder) shear, the elastic modulus was enhanced as the GO concentration increased, leading to higher degree of non-reciprocity. However, in this example, when the concentration exceeded 0.3 wt %, the degree of non-reciprocity declined even though the elastic modulus at right (harder) shear continued to increase. This was because the elastic modulus at left (softer) shear also increased, probably due to the deteriorated magnetic alignment degree of GO nanosheets in the gel as the result of more viscous pre-gel solution.

In addition, various samples serving as examples of the gel structure of the present invention were prepared under the same conditions except that different kinds of nanosheets were used, and the effects of differences in the nanosheets were investigated. Specifically, NR gel samples, which are examples of the gel structure of the present invention, were prepared using various nanofillers, including two-dimensional nanofillers such as titanium oxide, niobium oxide, and fluorohectorite nanosheets, as well as one-dimensional (1D) nanofillers such as boehmite nanofibers and carbon nanotubes. For each sample, the elastic modulus at strains of −20% (E_{L}) and +20% (E_{R}) was measured, and the degree of non-reciprocity (E_{R}/E_{L}) was calculated from the average tangent of the stress-strain curve. Every composite hydrogel showed a value of the degree of non-reciprocity (E_{R}/E_{L}) larger than 1, indicating that all of them responded to shear forces in an asymmetric manner. That is, it was found that mechanical asymmetric response could be obtained regardless of the nanosheet material. In other words, the E_{R}/E_{L} value is largely dependent on differences in the size, shape, and mechanical properties of the nanosheets, and it can be seen that by adjusting one or more of the size, shape, and mechanical properties of the nanosheets used, a gel structure that exhibits asymmetric response within an appropriate range depending on the application can be obtained.

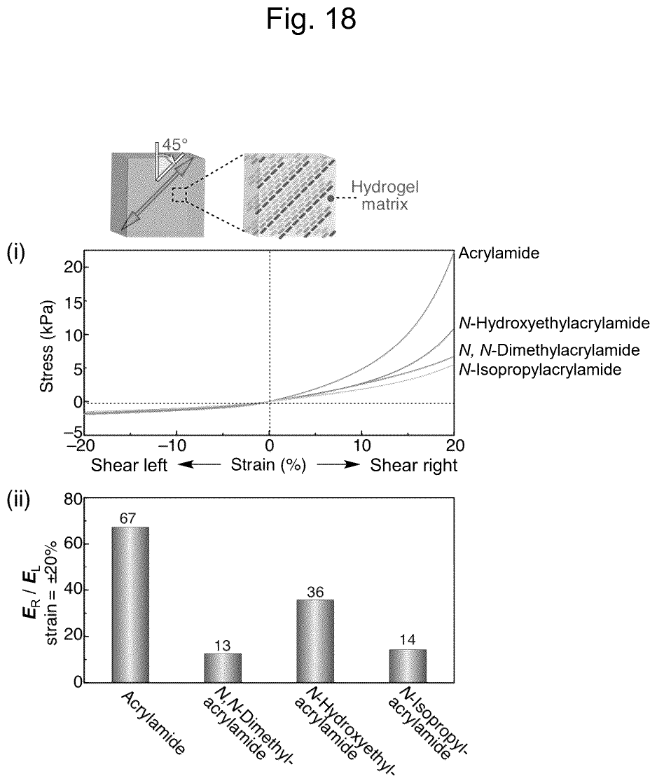

In addition, various samples serving as examples of the gel structure of the present invention were prepared under the same conditions, except that they were composed of different matrices, and the effects of these matrix differences were examined. The results are shown in FIG. 18. FIG. 18 shows the results from preparing NR gels composed of cross-linked polymers of acrylamide, N,N-dimethylacrylamide, N-hydroxyethyl acrylamide, and N-isopropylacrylamide as matrices to obtain stress-strain curves of −20% (E_{L}) and +20% (E_{R}), calculating the elastic modulus at strain from the average tangent, and calculating the degree of non-reciprocity (E_{R}/E_{L}). Every sample showed a value of the degree of non-reciprocity (E_{R}/E_{L}) larger than 1, indicating that all of them responded to shear forces in an asymmetric manner. It can also be seen that E_{R}/E_{L} varies depending on the matrix type. This is most likely because of the difference in their strength of interaction with the nanofillers embedded in the NR gel. That is, by appropriately combining the type of matrix and the type of anisotropic nanostructures, a gel structure with desired properties can be obtained.

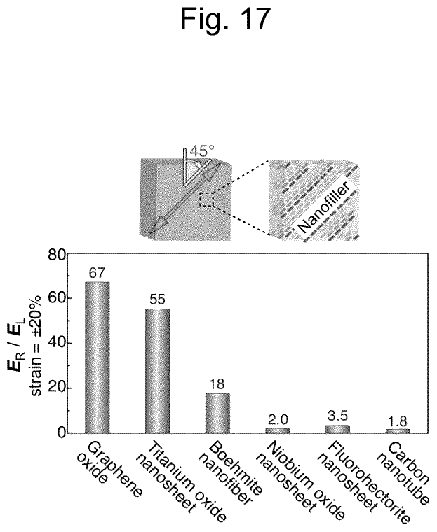

The above results suggest that by replacing GO nanosheets with other nanofillers (two-dimensional (2D) and even one-dimensional nanofillers) (FIG. 17) or polyacrylamide with another polymer (FIG. 18), similar hydrogels with asymmetric responses to shear can be prepared.

Furthermore, the gel structure of the present invention has notable features in its behavior when subjected to local deformation, in addition to global deformation for the whole gel. More specifically, the gel structure of the present invention deforms asymmetrically when a local force is applied thereto. This feature can induce the directional motion of objects with which the gel structure interacts. That is, the non-reciprocal mechanical system according to the present invention can induce directional mechanical energy. Specifically, the gel structure of the present invention in this example is a gel structure having at least a first and a second surface in a stationary state, wherein vibration energy applied from the first surface imparts directional mechanical energy (e.g., transport energy, rotational energy) to a solid or liquid disposed on at least a portion of the second surface as an output from the second surface.

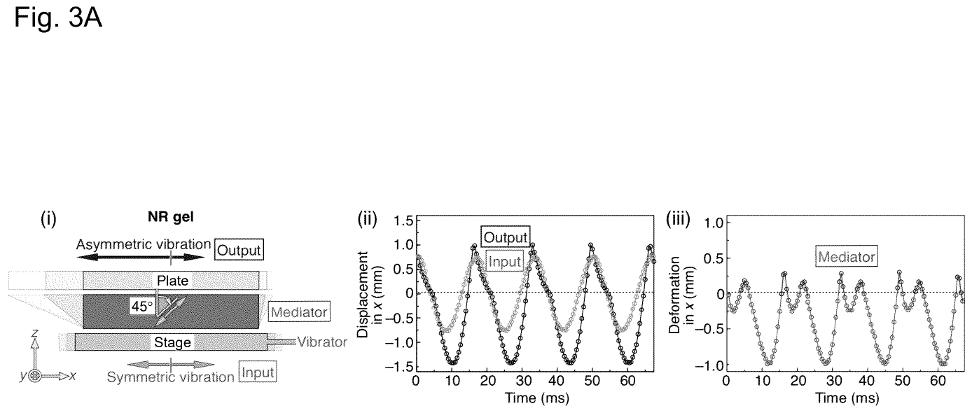

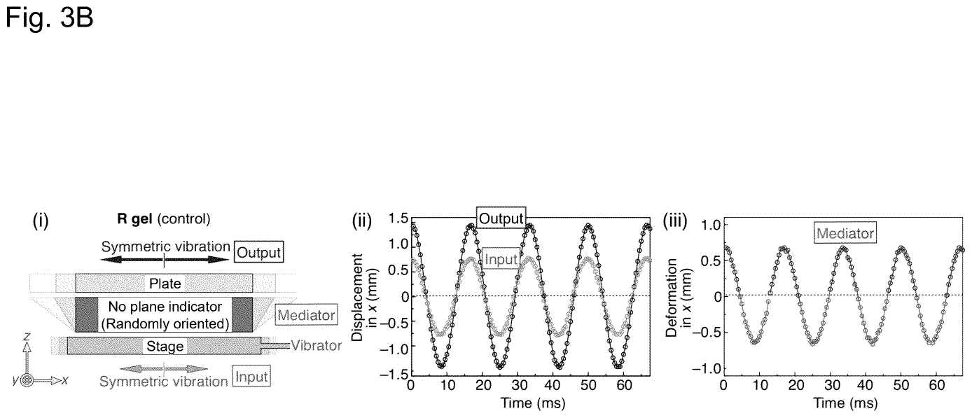

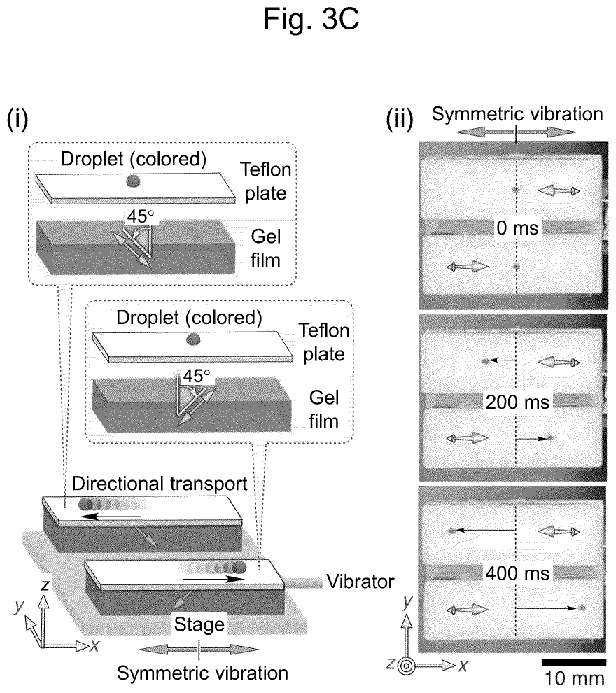

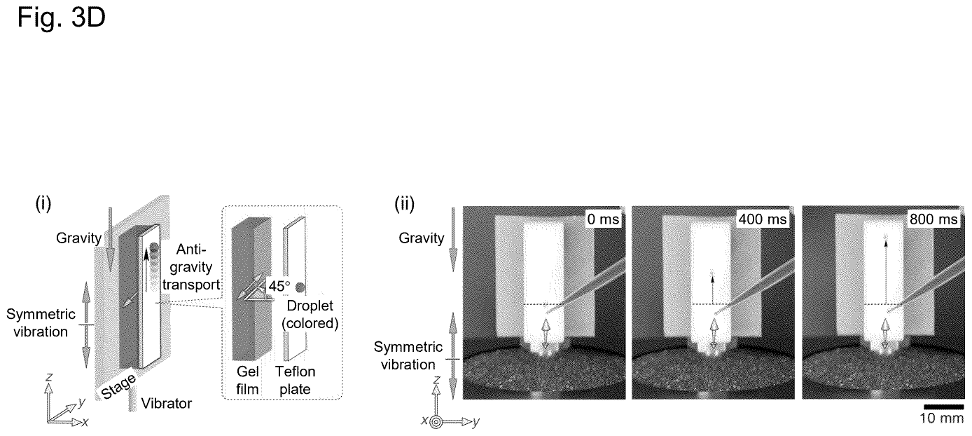

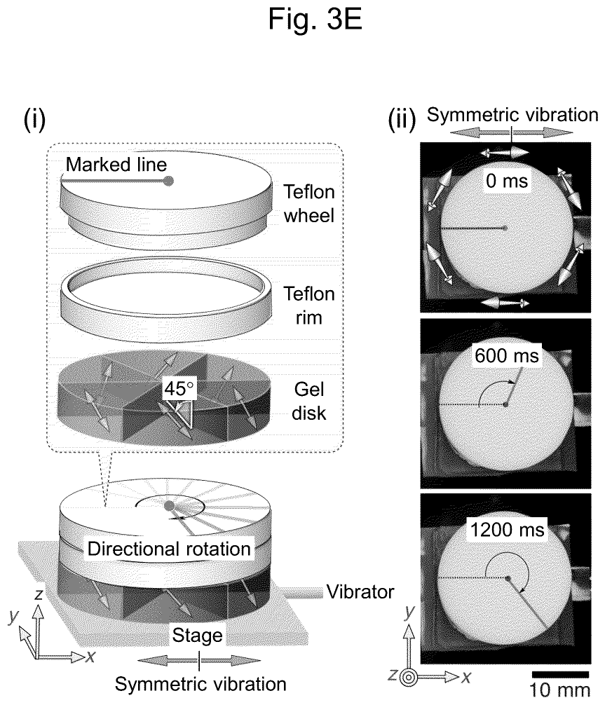

One example is shown in FIG. 3A. As shown in FIG. 3A, a machine-generated sinusoidal vibration (=input vibration) was applied to a NR gel film from the bottom, and the vibration at the top of the gel, which was pasted with a plate as loading, was monitored (=output vibration). When symmetric vibration (0.75 mm amplitude and 60 Hz frequency) was applied from the bottom (FIG. 3A, ii, input), a highly asymmetric vibration was afforded at the top (FIG. 3A, ii, output). This conversion was enabled by the vibrational shear deformation of the NR gel, which was highly asymmetric in terms of the frequency, amplitude, and peak shape (FIG. 3A, iii).

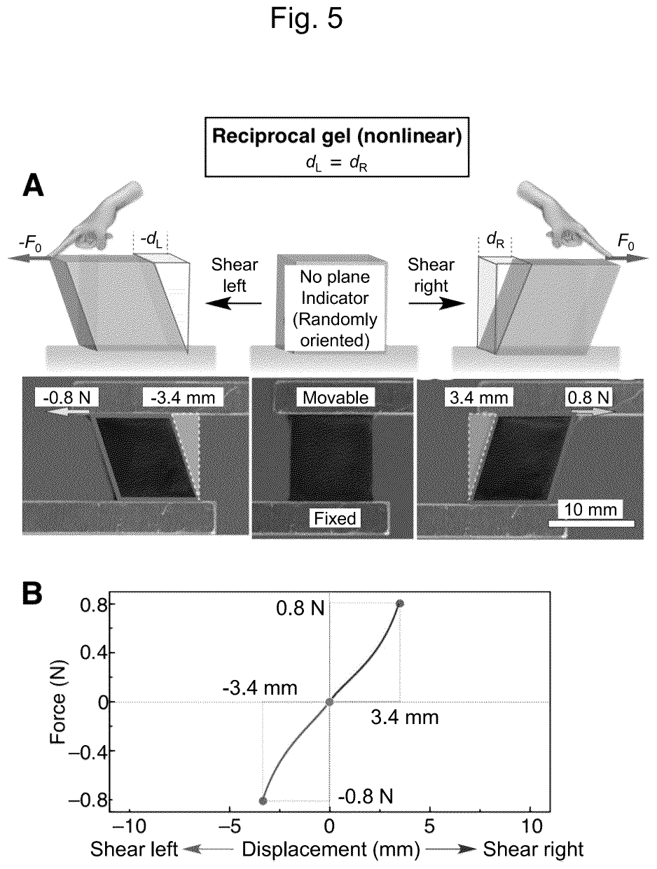

In contrast, a reciprocal gel containing randomly oriented GO nanosheets (R gel; FIG. 5) afforded symmetric vibration under the same conditions (FIG. 3B, ii, output) due to its symmetric deformation (FIG. 3B, iii). This result is consistent with the following: it is well known that when a composite elastomer consisting of a polymer matrix and nanofillers is deformed, the nanofillers align along the polymer chains, enhancing the mechanical performance of the elastomer, resulting in nonlinear elasticity (References 40); and several reports have confirmed that GO-polyacrylamide hydrogels exhibit nonlinear elasticity under tension and compression (References 41 to 44).

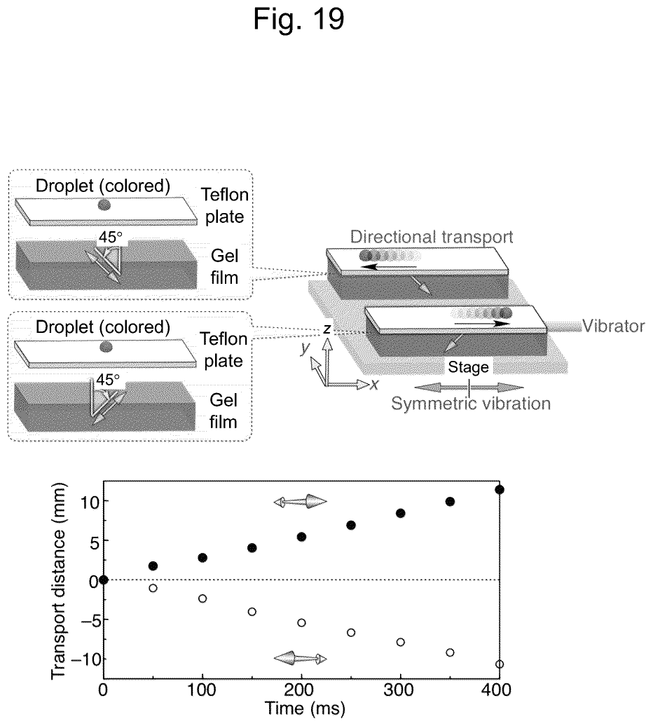

Using the asymmetric vibration generated above, efficient vectorial transport of objects can be realized, as exemplified by the water droplet transport (References 23). As shown in FIG. 3C (i), a NR gel film was placed on the vibration stage and covered with a Teflon (registered trademark) plate, on which a 0.3 μL droplet of water was placed. To exclude the effects of artifacts such as the tilt of the stage, another gel film with its harder direction opposite to the previous one, was also set on the same stage. When symmetric vibration was applied from the bottom, the water droplets started drifting unidirectionally toward the harder directions of the NR gel films (FIG. 19 and movie S3). When a horizontal symmetric vibration was applied from the sample stage, the water droplets unidirectionally drifted in the harder directions of the NR gel films with constant velocities of 26.6 mms⁻¹ and 28.5 mms⁻¹, respectively.

This unidirectional transport operated stably when the water droplets were continuously loaded (movie S3). As shown in FIG. 3D, the droplets could even climb up against gravity smoothly when the vibration stage was vertically arranged (movie S4).

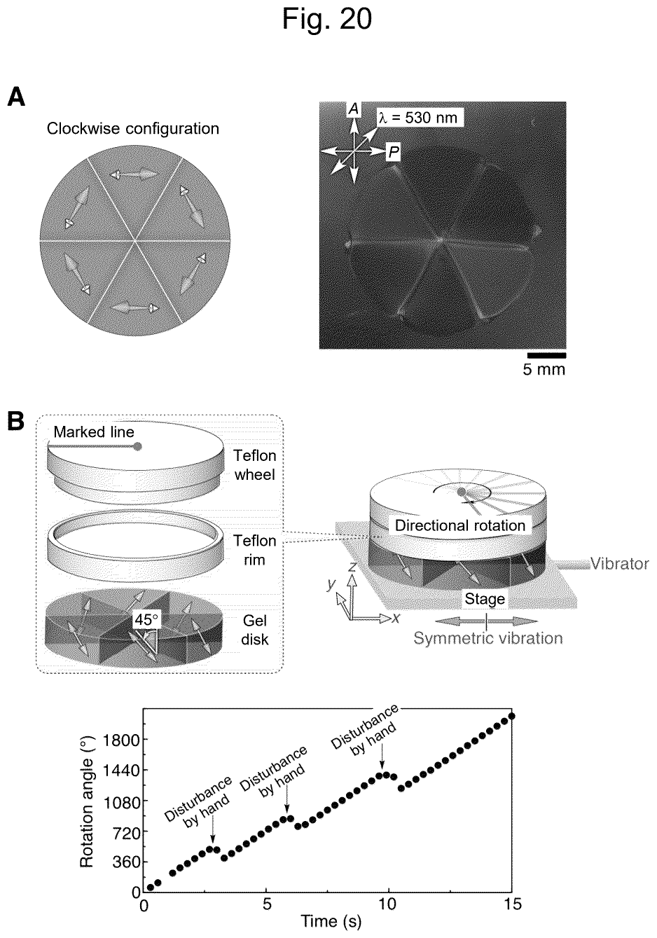

The NR gel of the present invention is also excellent in designability. Specifically, it can be processed into pieces with any shape or size, and such pieces can be assembled on demand. One example is shown in FIG. 3E. The present inventors developed a device by assembling six sector-shaped pieces of NR gels (details in FIG. 20) that could convert vibrational noises into rotational motion of a wheel loaded on the device (FIG. 3E and movie S5). Specifically, when a horizontal symmetric vibration was applied from the bottom, the sector gels vibrated asymmetrically and caused the clockwise rotation of the wheel with a constant velocity. The rotation device was quite stable and tolerant to external mechanical disturbances, and the rotation direction was controllable (FIG. 20 and movie S5). Importantly for potential application, the response of this rotation device was so robust that it could generate a regular rotational motion from noisy vibrations with irregular frequencies and small amplitudes, including those from an oil pump and a wireless speaker (movie S6), implying its promising use as an energy harvesting system.

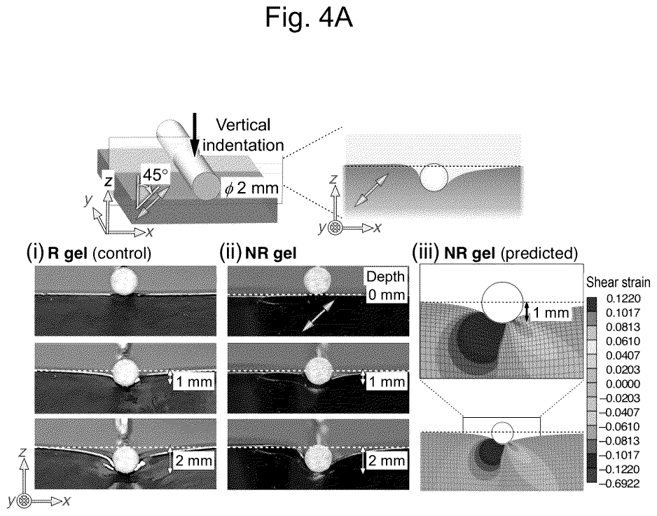

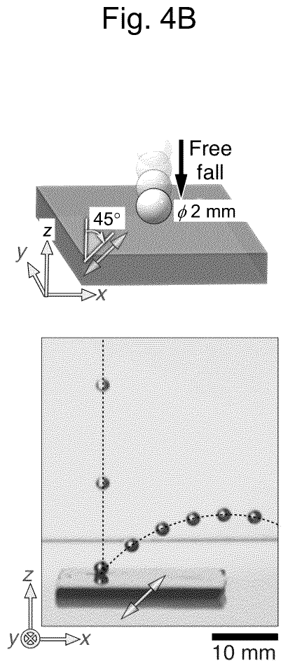

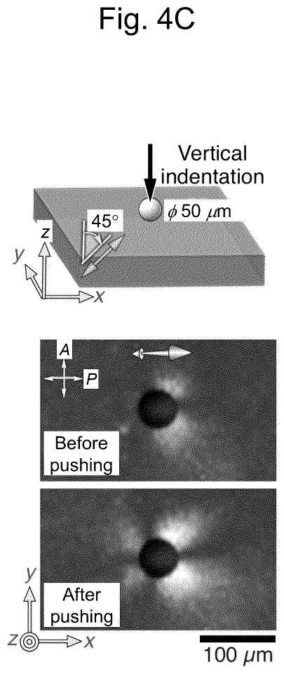

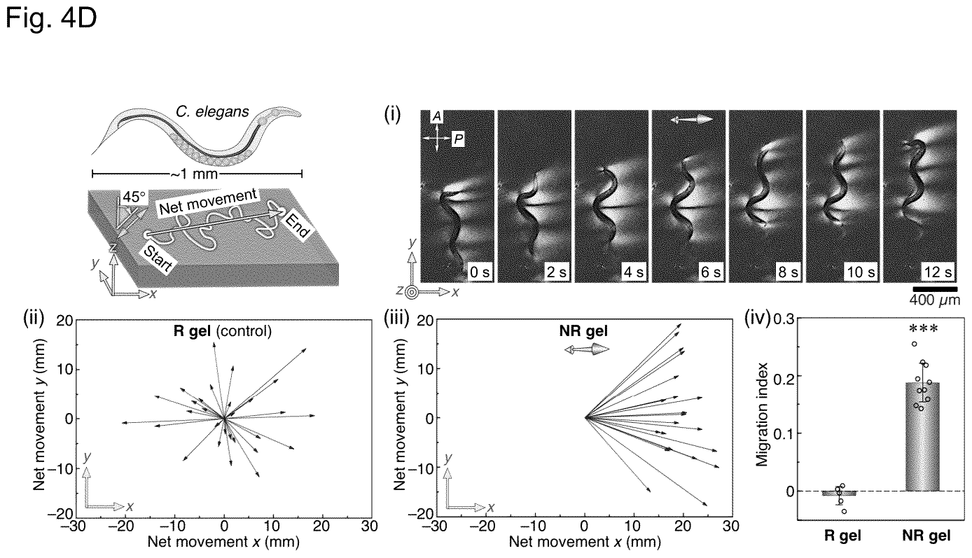

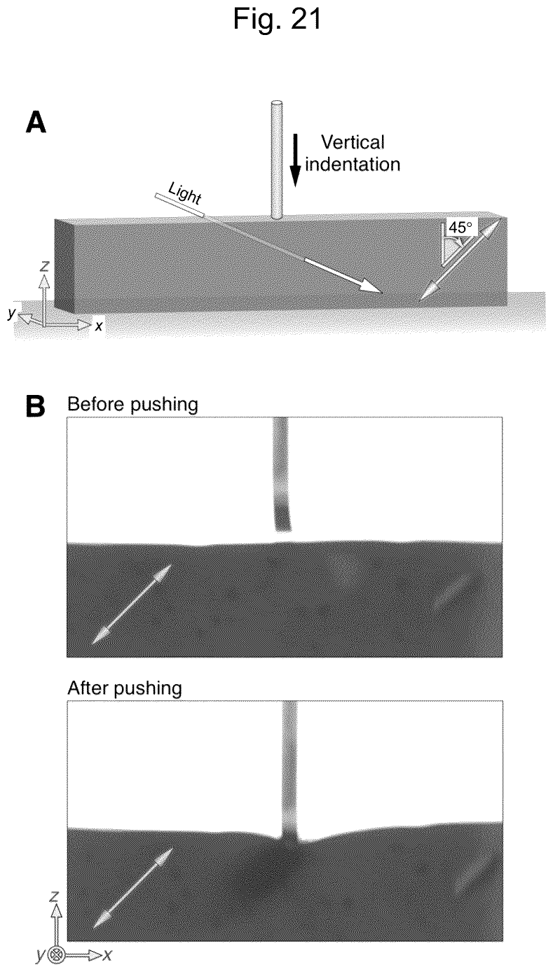

Next, it will be described that apart from global deformation for the whole gel, another notable feature of the NR gel is the behavior when subjected to local deformation. For example, when a φ2 mm metal cylinder laid parallel to the y-axis was pushed vertically onto the surface of the NR gel, it showed a highly asymmetric deformation (FIG. 4A, ii and movie S7), in contrast to the case of the R gel (FIG. 4A, i). At the left (softer) side of the cylinder, the gel bent sharply and contacted tightly with the cylinder. In contrast, at the right (harder) side, the gel bent gently, so that the deformation propagated over a wider range (FIG. 4A, ii). In the light transmission experiment (FIG. 21), a dark region was observed on the left (softer) side after the local compression. This indicates the localized buckling of GO nanosheets in this region. Such deformation can be attributed to the fact that the buckling occurred only at the left (softer) side.

The asymmetric deformation of the NR gel was consistent with the theoretical prediction based on finite element analysis (FIG. 4A, iii and movie S8; details in the Examples). This theoretical study also indicated a highly asymmetric distribution of elastic strains: the softer side showed much higher strain than the harder side. As described above, owing to such asymmetric deformation, the NR gel of the present invention can induce the directional motion of objects with which it interacts. For instance, when a φ2 mm metal ball was free-dropped onto a NR gel film, it always bounced to the harder side (FIG. 4B and movie S9).

Even micrometer-sized objects could also induce asymmetric deformation of the NR gel. To visualize such small deformations, polarized optical microscopy (POM) was used. When a 50 μm microbead was vertically pushed onto the gel, the bright area that emerged on the right (harder) side of the ball was much larger than that on the left (softer) side (FIG. 4C), indicating a highly asymmetric deformation.

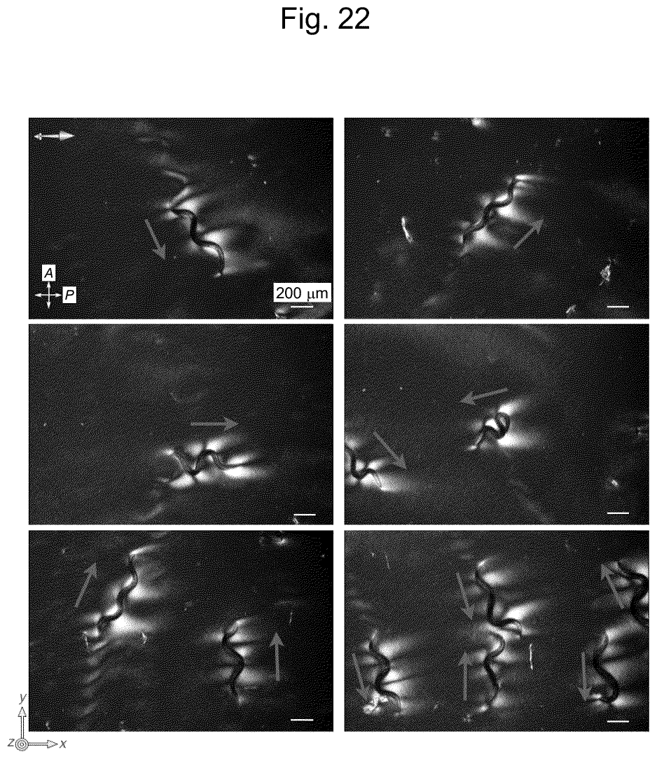

The present inventors attempted to use their NR gel to manipulate living creatures. Caenorhabditis elegans (C. elegans; FIG. 4D) was used. C. elegans is a type of nematode that has long attracted much attention as a model organism for studying animal development and behavior (References 24, 25). Indeed, when C. elegans crawled on the NR gel, an asymmetric deformation always occurred around the worm (FIG. 4D, i and FIG. 22), strongly implying that the migration of C. elegans might be biased by the NR gel.

To confirm this hypothesis, a NR gel film (NR gel was prepared using titanium oxide nanosheets instead of GO nanosheets to enhance the optical contrast of the POM images) was placed on a horizontal stage, and a group of C. elegans containing approximately 30 worms was transferred to the middle of the film, and then the worm migration was monitored for 50 minutes (FIG. 4D). Consequently, all the worms migrated to the right (harder) side and eventually gathered at the right end of the gel film (movie S10). As shown in the net movement of all the worms, the migration was certainly robustly biased to the right (FIG. 4D, iii). To exclude the effect of other possible stimuli such as light, odor, and the like, worm migration was also conducted simultaneously on two NR gels placed side by side with their harder directions pointing opposite to each other. As expected, C. elegans always migrated toward the harder side of the NR gels (movie S10). In contrast, when the R gel containing randomly oriented GO nanosheets was used instead, the worm migration was completely random (movie S10 and FIG. 4D, ii). For a more statistical study, the worm migration experiment was repeated 10 times for the NR gel, as well as 5 times for control, and the same results were observed with excellent reproducibility (FIG. 4D, iv). FIG. 22 shows POM images. The direction of the polarizing plate set between the backlight and the sample is denoted as the polarizer P, while that set between the sample and the detector is denoted as the analyzer A. The harder direction of the gel film was toward the right side, as depicted by the arrow in the upper left of FIG. 22. The moving direction of each worm is indicated by an arrow (→). It can be observed from these POM images that, regardless of the moving direction, all worms always experience the asymmetric deformation of the NR gel below them.

That is, one example of the present invention is a gel structure used as a culture medium for cells, microorganisms, or nematodes.

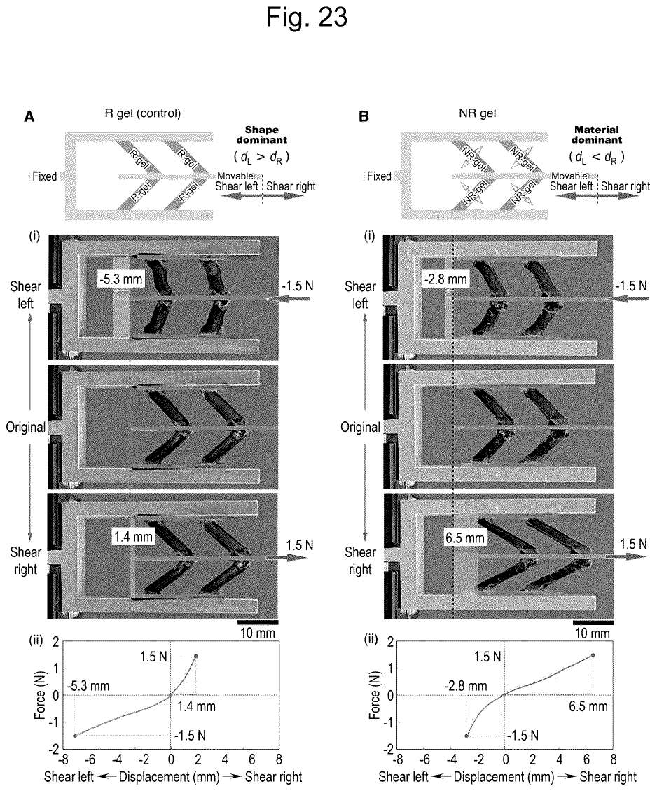

In summary, the present inventors developed a material that displays mechanical non-reciprocity, which exhibits asymmetric deformation to various types of forces. This material could induce the directional transport of a wide range of objects, from macroscopic ones to small living creatures, suggesting potential applications in various fields such as mechanics, energy, and biology. This material can also broaden the scope of current metamaterials systems and inspire more interesting designs that cannot be realized by current existing reciprocal materials (for example, see FIG. 23). FIG. 23 shows the results from preparing fishbone-structured frameworks by bridging a one side-open frame (unmovable) and a center plate (movable) with 4 parallelogram-shaped plates of the R gel (A) and the NR gel (B), and shearing the center plate by applying forces at the center plate toward left and light: (i) Optical images; (ii) graphs showing the relationship between the displacement and applied force. Note that in (B), the macroscopic shape of the framework apparently seems to exhibit higher resistance to the right-pulling force than the left-pulling force. However, the framework indeed showed higher resistance to the left-pulling force than the right-pulling force, because the asymmetry of the fishbone shape was overturned and reversed by the material deformation asymmetry in the NR gel.

In principle, the present design for non-reciprocal materials also applies to various kinds of elastomers other than hydrogels. One of the promising candidates is crosslinked liquid crystal elastomers, which is known to show large structural anisotropy. The material of the present inventors may pave the way toward practical applications of non-reciprocal mechanical systems and inspire the development of non-reciprocal materials in other physical systems, such as optics and acoustics.

### EXAMPLES

### 1. Materials and Methods

### 1.1 General and Materials

Unless otherwise noted, all commercial reagents were used as received. Deionized water was obtained from a Millipore model Milli-Q integral water purification system.

Acrylamide (AAm), N,N-Dimethylacrylamide, N-hydroxyethyl acrylamide, N-isopropyl-acrylamide, N,N′-methylenebisacrylamide (BIS), potassium persulfate (KPS), N,N,N′,N′-tetramethylethylenediamine (TMEDA), 2,2-diethoxyacetophenone, potassium dihydrogen phosphate (KH₂PO₄), disodium hydrogenphosphate (Na₂HPO₄), sodium chloride (NaCl), magnesium sulfate (MgSO₄), acetic acid, urea, cetyltrimethylammonium bromide and methyltrimethoxysilane are purchased from Wako Pure Chemical Industries. A water dispersion of graphene oxide (GO) with an average diameter of about 4 μm was purchased from NiSiNa Materials. Water dispersions of titanium oxide nanosheets and niobium oxide nanosheets were synthesized according to previous reports (26, 27). Aqueous dispersions of fluorohectorite nanosheets, boehmite nanofibers, and carbon nanotubes were kindly donated by Katakura & Co-op Agri, Kawaken Fine Chemicals, and KJ Specialty Pater, respectively. M9 buffer was prepared according to previous report (28).

### 1.2 Preparation of the Polyacrylamide Hydrogel (Reference Example)

AAm (0.6 g) and BIS (0.0064 g) were dissolved in deionized water (10 mL), and the resultant solution was degassed with N₂ and then soaked in an ice-water bath with stirring. To the solution was added TMEDA (5 μL) and a deionized-water solution of potassium persulfate (200 μL, 20 mgmL⁻¹) with stirring in the ice-water bath. From the resultant pre-gel solution, a portion (~4 mL) was poured into a glass cuvette (10 mm×10 mm×45 mm) in the ice-water bath, and the cuvette was transferred into a water bath at 25° C., so that free radical polymerization started. The mixture in the cuvette was left to stand in the water bath at 25° C. for 24 h to afford a cuboid of the polyacrylamide hydrogel, which was trimmed into appropriate shapes before experiments.

### 1.3 Preparation of the Reciprocal Gel (R Gel; Comparative Example) Containing Randomly Oriented GO Nanosheets

The pre-gel solution was prepared in the procedure similar to that of the polyacrylamide hydrogel (Section 1.2) except for using a water dispersion of GO nanosheets (0.3 wt %) in place of deionized water. The resultant pre-gel solution was subjected to the free radical polymerization in the procedure similar to that of the polyacrylamide hydrogel (Section 1.2) to afford a cuboid of the R gel, which was trimmed into appropriate shapes before experiments.

For preparing a film of the R gel, a hand-made glass cell (10 mm×10 mm×2 mm) was used in place of a glass cuvette (10 mm×10 mm×45 mm).

### 1.4 Preparation of the Non-Reciprocal Gel (NR Gel; Examples of the Present Invention)

The pre-gel solution was prepared in the procedure similar to that of the R gel (Section 1.3), from which a portion (~4 mL) was poured into a glass cuvette (10 mm×10 mm×45 mm). The cuvette was sealed and contained in a polystyrene box filled with ice water in such a way that the faces of the ice box became parallel to those of the cuvette. The ice box was placed on a horizontal rotating stage with a spacer adopting a corner angle of 45°, which was equipped in a φ 100 mm horizontal bore of a 10 T superconducting magnet (JASTEC model JMTD-10T100) (FIG. 24). The stage was kept rotating for 24 h, where the temperature inside the ice box was gradually raised and finally reached to room temperature, so that the free radical polymerization proceeded to afford a cuboid of the NR gel with a tilt angle θ of 45°. For changing θ, the corner angle of the spacer was tuned accordingly. Note that the sample rotation is indispensable for achieving unidirectional alignment. GO nanosheet is known to align parallel to the magnetic field, where GO nanosheets have a freedom of rotation around the field. Therefore, if the sample is fixed, the normal vectors of GO nanosheets distribute randomly in the plane perpendicular to the field. Meanwhile, if the sample is rotated around an axis perpendicular to the field (see FIG. 6), GO nanosheets direct their normal vectors parallel to the rotation axis, so that GO nanosheets align unidirectionally (29).

For preparing a film of the NR gel, a hand-made glass cell (10 mm×10 mm×2 mm) was used in place of a glass cuvette (10 mm×10 mm×45 mm).

For preparing the NR gel in FIG. 15, GO nanosheets were chopped into smaller nanosheets by sonicating their water dispersion (0.5 wt %) with a QSONICA model XL-2000-600 homogenizer for 2 min. The average size of the resultant chopped GO nanosheets was estimated by transmission electron microscopy (TEM) observation using a JEOL model JEM-1230 transmission electron microscope operating at an anode voltage of 80 kV.

### 1.5 Shear Test

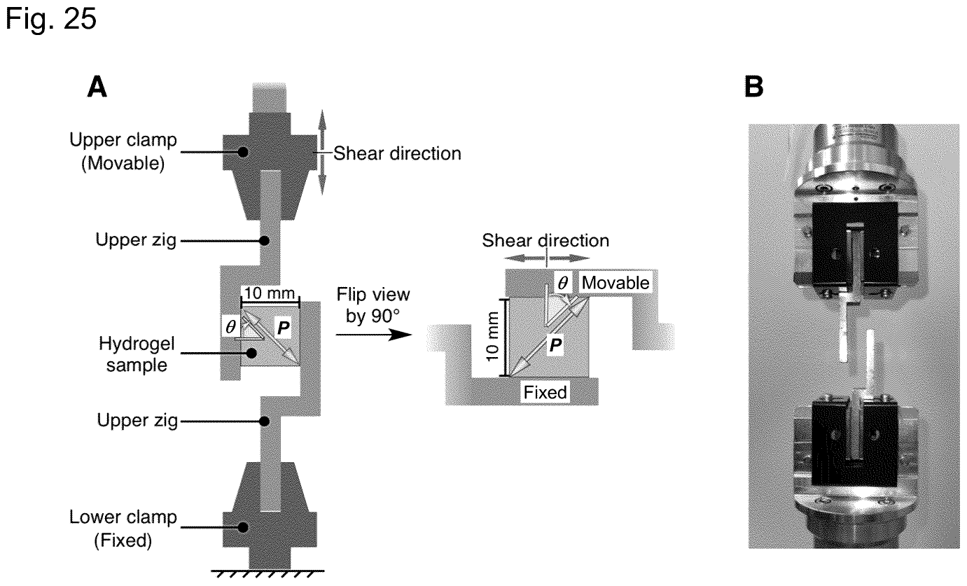

Shear tests were carried out at room temperature by using a Shimadzu model EZ test machine equipped with hand-made zigs (FIG. 25). A cube-shaped (10 mm×10 mm×10 mm) sample was glued to a pair of double-L shaped zigs at its two faces, where one of them was clamped at the unmovable bottom grip, while the other zig was clamped at the movable top grip that was connected to a 50 N load cell (FIG. 25A). The top grip was moved up and down to apply shear forces to the sample. In the force-scanning test (FIG. 1, A and D, FIGS. 5, 8, and 23, and movie S1), the top grip was displaced in one way with gradually changing the force from 0 to −0.8 N or from 0 to +0.8 N at a constant rate of 0.02 Ns⁻¹. The force-scanning test with applying shear forces at a single point (FIG. 8) was also conducted under the same conditions except for connecting the upper face of the cube-shaped gel sample and the double-L-shaped zig via a 1 mm-thick bar. In the repetitive cycle test (FIG. 9), the top grip was displaced in an oscillatory manner with gradually changing the force between −0.8 to +0.8 N over 100 cycles. In the conventional strain-stress curve measurement (FIG. 2A and FIGS. 13-18), the top grip was displaced in one way with gradually changing the strain from 0 to −2.0 mm or from 0 to +2.0 mm at a constant rate of 0.1 mm s⁻¹. Elastic moduli were calculated as the average tangent in the stress-strain curves.

For shear test of fish bone-shaped frameworks (FIG. 23), four parallelogram-shaped plates (45° and 135° angles, 5 mm×14 mm sides, 10 mm thick) of the R- or NR-gel were prepared according to the procedure in Section 1.4 using a hand-made mold. A one side-open frame (70×30 mm whole size, 4 mm frame width, 12 mm frame thickness) and a center plate (76 mm×26 mm×1 mm) were prepared using an aluminum plate and a glass plate, respectively. The center plate was inserted at the middle of the one side-open frame, and the plate and frame were bridged with the parallelogram-shaped gel plates (two gel plates for each of the upper and lower regions) with pasting their interfaces. See also FIG. 23. The one side-opened frame was clamped at the unmovable bottom grip of the test machine, while the center plate was clamped at the movable top grip. The top grip was displaced in one way with gradually changing the force a from 0 to −1.5 N or from 0 to +1.5 N at a constant rate of 0.02 N s⁻¹.

### 1.6 Light Transmittance Measurement

A cube (10 mm×10 mm×10 mm) of the NR gel was placed on a Shimadzu model EZ test machine with the same configuration as the shear deformation test (Section 1.5 and FIG. 17). A light from a LED cold light source (Olympus, KL 1600) was irradiated at the center of the sample in the configuration of FIG. 2B, where the incident light was directed parallel to the y-axis. Changes in the light transmittance of the sample during the shear deformation were recorded with a SONY model α7c digital camera with a Tamron model 28-200 mm F2.8-5.6 lens. The intensity of transmitted light was quantified by analyzing the recorded photos using software ImageJ (30). It is known that the light absorption of GO nanosheets is highly dependent on the angle between GO plane and light path. The original NR gel showed a considerable light transmittance since all GO nanosheets exposed their edges to the light source. When the shear deformation of the gel induced the buckling of GO nanosheets to reduce their orientation order, the light transmittance of the gel was decreased, so that the region where the buckling took place could be visualized. The similar technique was also used for the local compression experiment (FIG. 21).

### 1.7 Small-Angle X-Ray Scattering (SAXS) Measurement

SAXS measurements were carried out by a. Rigaku NANOPIX SAXS/WAXS measurement system using a Rigaku model HyPix-6000 detector. The scattering vector q (q=4π sinθ/λ; θ=scattering angle; λ=wavelength of the incident X-ray beam [1.54 Å]) and position of the incident X-ray beam on the detector were calibrated using several orders of layer reflections from silver behenate (d=58.380 Å). The sample-to-detector distance was set at 1.4 m. A sample of the NR gel was sliced into a 2 mm-thick film just before the SAXS measurement and exposed to the X-ray beam in the configuration of FIG. 2B, where the incident X-ray was directed parallel to the y-axis. The acquired scattering 2D images were integrated along the Debye-Scherrer ring by using 2D data processing software Rigaku 2DP (31), affording the corresponding 1D profiles. Intensity of the 1D SAXS profiles was normalized by the exposure time.

### 1.8 Scanning Electron Microscopy (SEM) Measurement

According to the reported procedure (3.2), a 2 mm-thick film of the NR gel was fixed by in-situ silica condensation and then dried, which was used as the sample for the original state. The samples of the left-sheared and right-sheared states were also prepared in the same procedure, except for applying the left and right shear during the solidification step, respectively. The resultant samples were cut perpendicular to the film surfaces, and their cross sections were observed by SEM using a Hitachi model SU8010 field emission scanning electron microscope.

### 1.9 Vibration Conversion

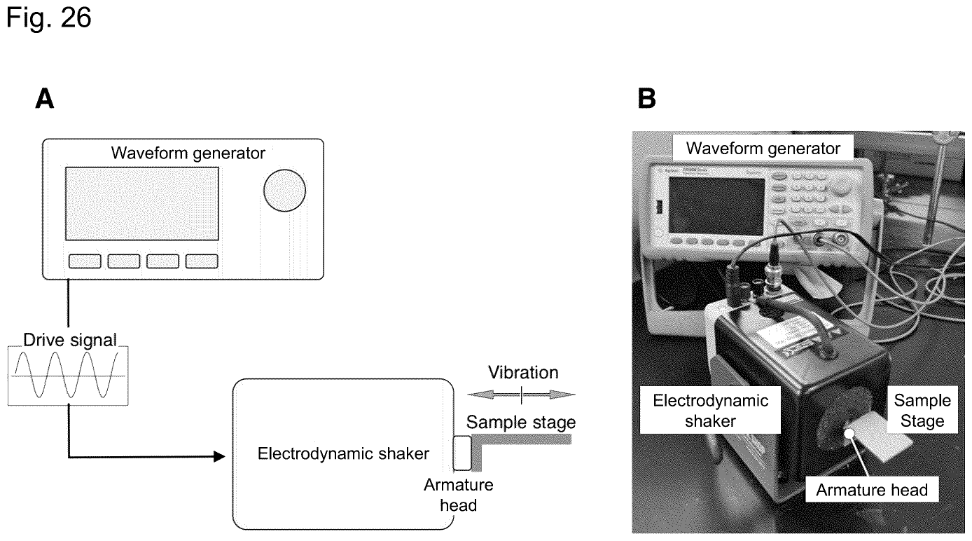

The setup for the vibration conversion experiment was illustrated in FIG. 26. Mechanical vibration was generated by a Modal Shop model K2007E01 electrodynamic shaker, which was controlled by an Agilent model 33500B Series waveform generator. A sample stage was fixed to the armature head of the shaker in such a way that the stage became perfectly horizontal and vibrated along the x-axis. When the waveform generator applied a sinusoidal function as the drive signal to the shaker, the stage sinusoidally vibrated with constant amplitude and frequency, where these parameters could be changed by tuning the drive signal.

At the center of the sample stage, a film of gel (30 mm×10 mm×2 mm) prepared in the procedure described in Sections 1.3 and 1.4 was pasted with directing its 30 mm side parallel to the vibration direction (=x-axis). In the case of the NR gel, the film was prepared in such a way that the plane indicator P (FIG. 7) was directed perpendicular to the 10 mm side of the film. The top surface of the sample film was pasted with a Teflon plate to serve as mass load. Then a sinusoidal function from the waveform generator was applied to the shaker, so that the sample stage underwent asymmetric vibration. The side view video of the whole setup was taken by a NAC model MEMRECAM Q2m high-speed camera at a frame rate of 2000 s⁻¹. The time course profiles of the input vibration at the bottom and that of the output vibration at the top were obtained by determining the positions of the sample stage and the Teflon plate, respectively, frame by frame using software Image J (30).

### 1.10 Directional Transport of Droplets

The shaker, the waveform generator, the sample stage, the film (30 mm×10 mm×2 mm) of the NR gel, and the Teflon plate were arranged into the same setup as that in Section 1.9. Then a sinusoidal function from the waveform generator was applied to the shaker, so that the sample stage underwent asymmetric vibration. Before or during this vibration, 0.3 μL water droplets colored with Rhodamine B for visibility were loaded on the Teflon plate. For consecutive loading of water droplets, a Chemyx model Fusion 100 syringe pump was used. The top view video of the sample stage was taken by an Apple model iPhone 13 smartphone. The time course profiles of the transport distances of droplets were obtained by determining the positions of droplets frame by frame using software Image J (′30).

### 1.11 Directional Rotation of a Wheel

A 2 mm-thick film of the NR gel was trimmed into a sector with a radius of 12 mm and a central angle of 60° in such a way that the plane indicator P (FIG. 7) was directed perpendicular to the bisectrix of the two straight sides of the sector (see FIG. 20A). Six pieces of such sectors were prepared and assembled into a full circular disk with a diameter of 24 mm, in which the harder directions of the sectors were arranged clockwise or counterclockwise.

The shaker, the waveform generator, and the sample stage were arranged into the same setup as that in Section 1.9. At the center of the sample stage, the gel disk prepared as above was pasted. The top surface of the gel disk was pasted with a Teflon dish to act as the rim for the wheel. A Teflon disk (21.5 mm diameter and 2.0 mm thick) was placed onto the Teflon dish to act as the wheel. Then a sinusoidal function from the waveform generator was applied to the shaker, so that the sample stage underwent symmetric vibration. The top view video of the sample stage was taken by an Apple model iPhone 13 smartphone. The time course profiles of the rotation of the wheel were obtained by determining the angle of the line marked on the wheel frame by frame using software Image J (30).

For the demonstration of energy harvesting, the gel disk was pasted on a glass plate at its bottom and then pasted at its top with the Teflon dish that accommodated the Teflon disk. This assembly was then pasted at its bottom onto an oil pump (ULVAC, GCD-051X) or a wireless speaker (EWA, A106 Pro). For the demonstration using the wireless speaker, a 130 Hz test tone was played.

### 1.12 Directional Bouncing of Free-Dropped Ball

A 2 mm-thick film of the NR gel was placed on a horizontal flat stage. A φ 2 mm metal ball was released from an electromagnet above the hydrogel film and allowed to free-drop onto the hydrogel film. The side view video of the dropping and bouncing of the ball was recorded by a NAC model MEMRECAM Q2m high-speed camera. To exclude the possibility of artifacts, the R gel containing randomly oriented GO nanosheets was used as a control sample. Also, the experiments for the NR gel were conducted twice with its harder direction pointing to the right and left sides, respectively, relative to the camera view.

### 1.13 Polarized Optical Microscopy (POM) Observation

For visualizing the deformed region of the NR gel under local compression with small objects, the POM technique was used. A 2-mm thick film of the NR gel was set to a Nikon model Eclipse LV100POL optical polarizing microscope, where the polarizer and analyzer were set parallel and perpendicular to the x-axis (=film's optical axis). For enhancing optical contrast, the NR gel was prepared with titanium oxide nanosheets instead of GO nanosheets. φ 50 μm microbeads were placed on the surface of the gel film, which was covered with a cover glass. Then the cover glass was vertically pushed, so that the vertical indentation of the microbeads took place. In this configuration, only deformed regions exhibited bright appearance due to the directional discrepancy between the film's optical axis and the polarizer. The similar technique was also used for observing the crawling of C. elegans (FIG. 22).

1.14 Directional Migration of C. elegans

C. elegans strain and culture: The N₂ Bristol strain was used for all experiments. C. elegans was cultured at 20° C. on E. coli OP50 according to standard methods in literature (33).

Preparation of gel films: A film of the NR gel (40 mm×30 mm×2 mm) was prepared according to the procedure in Section 1.4. As a control sample, a film of the R gel containing randomly oriented GO nanosheets was also prepared according to the procedure in Section 1.3. The gel films were soaked in an excess amount of M9 buffer (28) overnight to full exchange of the liquid medium before use.

C. elegans behavior assay: Developmentally synchronized L4 hermaphrodite worms were transferred to NGM plates containing 1.5 wt % GO nanosheets 1 day before assaying. Worms were washed 3 times with M9 buffer and then placed on the film of the NR gel on the aluminum plate. A cover glass (Matsunami. No. 1) was put on top of the gel film for enhancing the interaction between worms and the gel film. The temperature of the aluminum plate was controlled with a Peltier thermoelectric temperature controller (VICS, UT-4040CE-M and VTH-1800FA) to establish and maintain 20° C. during the assay. The temperature of the gel film was measured with a two-probe digital temperature logger (Hioki, LR5021). Worm migration was recorded with a CCD camera (Azure. EMVC-CB640M3) at a rate of 2 Hz for 50 minutes. Videos were analyzed using custom written scripts in Python. Animal movement was marked using tierpsy-tracker (34). The migration index was calculated for 40 minutes of each video, with excluding the first and the last five minutes. Statistical analysis was performed with R software. Custom scripts are available upon request.

### 2. Supplementary Text

### 2.1 Mechanism for the Buckling of GO Nanosheets and the Emergence of Mechanical Non-Reciprocity

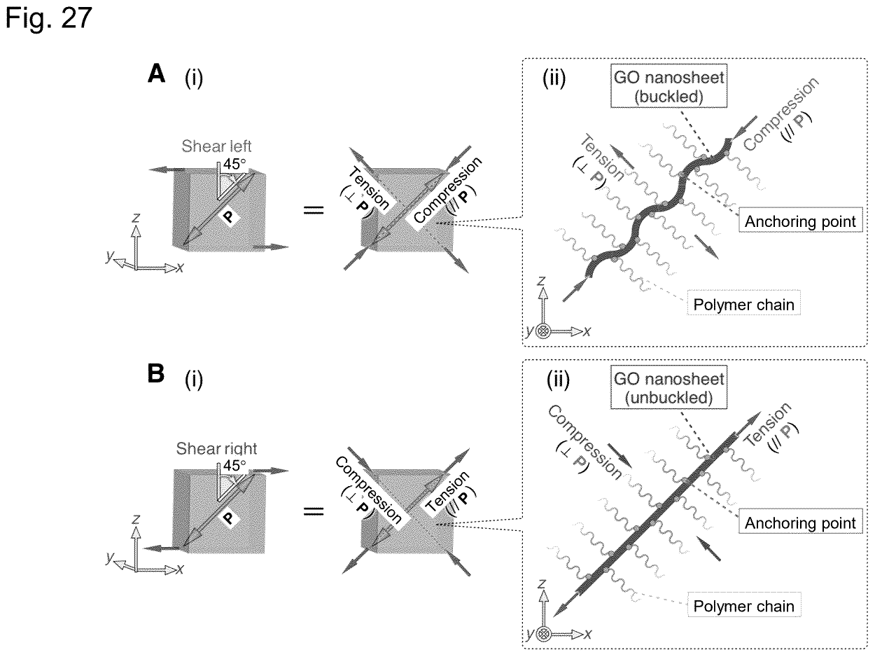

The matrix of the NR gel consists of a hydrogel composed of a polyacrylamide network, which is characteristic of its small mesh size (~10 nm) (35) and abundant hydrogen-bonding sites capable of binding strongly to GO nanosheets (36). Because a GO nanosheet is laterally wide (~4 μm), a large number of polymer chains in the network are anchored on a GO nanosheet through multivalent hydrogen bonds (FIG. 27). This anchoring between polymer chains and nanosheets can effectively enhance the mechanical properties since it can increase the number of crosslinking points for the hydrogel network and facilitate the load transfer from the polymer matrix to the ultrahard GO (Young's modulus=~200 GPa) (37). However, such reinforcement works only when GO nanosheets experience an in-plane stretch force. Due to its ultrathin shape, a GO nanosheet can buckle upon the in-plane compression even at a tiny strain (~0.001%), as predicted by the Euler's buckling formula ε=(π²/3)(h/λ)²=~10⁻⁵, where λ is the in-plane compression strain necessary for cause the buckling of a GO nanosheet, h is the thickness of GO nanosheet (~1 nm), and A is the pitch of buckling (~300 nm; estimated from the SEM images in FIG. 10). Once buckled, the apparent modulus of GO nanosheets drops drastically and the extra crosslinking points due to the polymer anchoring fail to work, so that the contribution of GO nanosheets to the mechanical properties vanishes quickly.

When the NR gel is sheared to the left, which is equivalent to the sum of tension perpendicular to the GO plane and compression parallel to the GO plane (FIG. 27A), GO nanosheets readily buckle due to the in-plane compression and no longer serve as reinforcers. In contrast, the shear to the right is equivalent to the sum of tension parallel to the GO plane and compression perpendicular to the GO plane (FIG. 27B), so that GO nanosheets hardly buckle and retain serving as reinforcers. Such a difference in the reinforcement ability of GO nanosheets under the left and right shear causes the non-reciprocity.

The above proposed mechanism is in good agreement with our experimental observations. According to the mechanism, once buckled, GO nanosheets cannot serve as reinforcers, so that the shear response toward left (softer) side is not sensitive to the conditions (concentration and size) of GO as long as they are well aligned (FIGS. 15 and 16). In contrast, the shear response toward right (harder) side was largely affected by the conditions (concentration and size) of GO (FIGS. 15 and 16), as well as the interaction between GO and the polymer matrix (FIG. 18).

### 2.2 Development and Application of the Macroscopic Non-Reciprocal Elastic Model

### 2.2.1 Development of the Non-Reciprocal Elastic Model

The theoretical prediction by the finite element analysis (FIG. 4A, iii) was obtained using the finite element package ABAQUS (38). The user-defined material subroutine UMAT was used to implement the non-reciprocal elastic model of the NR gel, which was developed by extending an orthotropic form of linear elasticity.

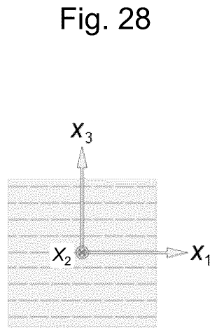

Linear elasticity in an orthotropic material is defined by giving the engineering constants (38, 39): the Young's moduli Eᵢ (i=1,2,3); the Poisson's ratios vᵢⱼ (i=1,2,3, j=1,2,3, i+j); and the shear moduli G₁₂, G₂₃, and G₁₃ associated with the material's principal directions (FIG. 28). These engineering constants define the elastic compliance matrix

$$\{ \begin{matrix}Δ {ε}_{11} \\ Δ {ε}_{22} \\ Δ {ε}_{33} \\ 2 Δ {ε}_{12} \\ 2 Δ {ε}_{23} \\ 2 Δ {ε}_{31}\end{matrix} \} = [ \begin{matrix}1 / {E}_{1} & - {υ}_{21} / {E}_{2} & - {υ}_{31} / {E}_{3} & 0 & 0 & 0 \\ - {υ}_{12} / {E}_{1} & 1 / {E}_{2} & - {υ}_{32} / {E}_{3} & 0 & 0 & 0 \\ - {υ}_{13} / {E}_{1} & - {υ}_{23} / {E}_{2} & 1 / {E}_{3} & 0 & 0 & 0 \\ 0 & 0 & 0 & 1 / {G}_{12} & 0 & 0 \\ 0 & 0 & 0 & 0 & 1 / {G}_{23} & 0 \\ 0 & 0 & 0 & 0 & 0 & 1 / {G}_{31}\end{matrix} ] \{ \begin{matrix}Δ {σ}_{11} \\ Δ {σ}_{22} \\ Δ {σ}_{33} \\ Δ {σ}_{12} \\ Δ {σ}_{23} \\ Δ {σ}_{31}\end{matrix} \} , \qquad ( S1 )$$

where Δεᵢⱼ(i=1,2,3, j=1,2,3) and Δσᵢⱼ (i=1,2,3, j=1,2,3) are the increments of strain and stress, respectively. The Poisson's ratio vᵢⱼ characterizes the transverse strain in the xⱼ-direction when the material is subjected to the uniaxial stress in the xᵢ-direction. The present inventors considered the elastic compliance matrix to be asymmetric to develop the non-reciprocal elastic model.

Because the non-reciprocity of the elastic compliance matrix highly increased the computational costs, the present inventors reduced eq. S1 to the two-dimensional case under plane strain conditions

$$Δ {ε}_{22} = 0 , Δ {ε}_{12} = 0 , and Δ {ε}_{23} = 0 , \qquad ( S2 )$$

leading to

$$\{ \begin{matrix}Δ {ε}_{11} \\ Δ {ε}_{33} \\ 2 Δ {ε}_{31}\end{matrix} \} = [ \begin{matrix}( 1 - {υ}_{12}^{2} ) / {E}_{1} & - ( 1 + {υ}_{12} ) {υ}_{31} / {E}_{3} & 0 \\ - ( 1 + {υ}_{12} ) {υ}_{13} / {E}_{1} & ( 1 - {υ}_{13} {υ}_{31} ) / {E}_{3} & 0 \\ 0 & 0 & 1 / {G}_{31}\end{matrix} ] \{ \begin{matrix}Δ {σ}_{11} \\ Δ {σ}_{33} \\ Δ {σ}_{31}\end{matrix} \} , \qquad ( S3 )$$

which was derived using v₁₂=v₂₁, v₃₁=v₃₂, and v₁₃=v₂₃ due to the geometrical symmetry in the x₁- and x₂-directions. Eq. S3 included the engineering constants, E₁, E₃, v₁₂, v₁₃, v₃₁, and G₃₁, which were modeled and determined from experiments below.

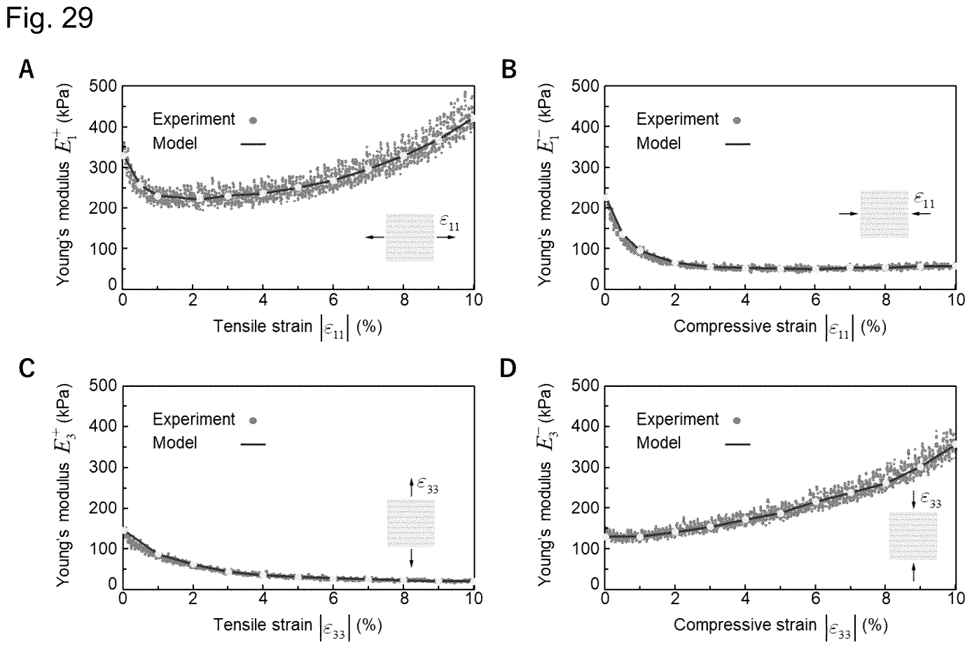

FIG. 29 shows the Young's moduli, E₁ and E₃, under uniaxial tension and compression in the x₁- and x₃-directions, respectively. The Young's moduli were estimated as the instantaneous gradient in the experimental stress-strain response so that they were found to be nonlinear as a function of the corresponding strain. Thus, the Young's moduli were modeled as

$${E}_{1} = \{ \begin{matrix}{E}_{1}^{+} ( \vert {ε}_{11} \vert ) & if {ε}_{11} \geq 0 \\ {E}_{1}^{-} \vert ( {ε}_{11} \vert ) & if {ε}_{11} < 0\end{matrix} , \qquad ( S4 )$$

$${E}_{3} = \{ \begin{matrix}{E}_{3}^{+} ( \vert {ε}_{33} \vert ) & if {ε}_{33} \geq 0 \\ {E}_{3}^{-} ( \vert {ε}_{33} \vert ) & if {ε}_{33} < 0\end{matrix} . \qquad ( S5 )$$

The nonlinear responses of E₁^{+,-}(|ε₁₁|) and E₃^{+,-}(|ε₃₃|) were modeled by a piecewise linear function of |ε₁₁| and |ε₃₃|, respectively (FIG. 29). The piecewise linear functions were determined by the experimental data in 0%≤|ε_{11,33}|≤10%, and were approximated by extrapolation outside of the range. Tension and compression asymmetry appeared especially at the tension and compression responses of E₁^{+,-}(|ε₁₁|). The response of E₁⁺(|ε₁₁|)>E₁⁻(|δε₁₁|) and E₃⁻(|ε₃₃|)>E₃⁺(|ε₃₃|) are reasonable because the nanofillers in the NR gel buckle under the condition of ε₁₁<0 and ε₃₃ ≥0. The case of ε₁₁<0 is trivial, whereas the buckling under ε₃₃≥0 can be interpreted by the Poisson's effect.

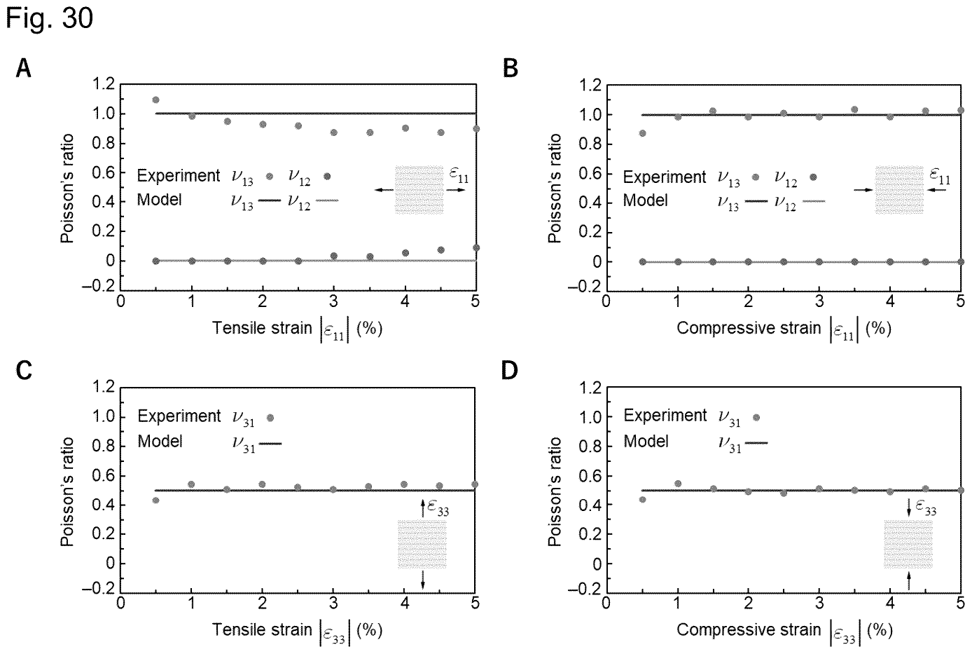

The Poisson's ratios were approximately constants (FIG. 30) and were independent of tension and compression. The Poisson's ratios were modeled as

$${v}_{12} = 0 , {v}_{13} = 0.98 , and {v}_{31} = 0.49 , \qquad ( S6 )$$

to avoid the singularity caused by the material incompressibility when 1−v₁₂−v₁₃=0 and 1−v₃₁−v₃₂=1−2v₃₁=0.

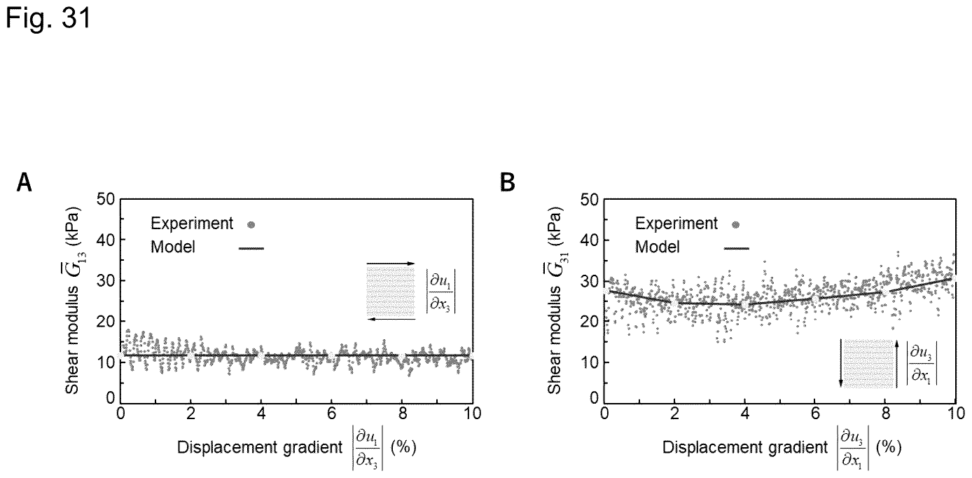

FIG. 31 shows the two different responses of

$${\overline{G}}_{13} and {\overline{G}}_{31}$$

under the simple shear test prescribed by the displacement gradients, du₁/dx₃ and du₃/dx₁, respectively. Here

$${\overline{G}}_{13} ( \vert {du}_{1} / {dx}_{3} \vert ) = {\overline{G}}_{13} ( - \vert {du}_{1} / {dx}_{3} \vert ) and {\overline{G}}_{31} ( \vert {du}_{3} / {dx}_{1} \vert ) = {\overline{G}}_{31} ( - \vert {du}_{3} / {dx}_{1} \vert )$$

because of the geometrical symmetry in the positive and negative directions of the x₁- and x₃-axes.

The nonlinear responses of

$${\overline{G}}_{13} ( \vert {du}_{1} / {dx}_{3} \vert ) and {\overline{G}}_{31} ( \vert {du}_{3} / {dx}_{1} \vert )$$

were also modeled using the piecewise linear functions in the non-reciprocal elastic model.

The framework of linear elasticity does not distinguish

$${\overline{G}}_{13}$$

and

$${\overline{G}}_{31} .$$

Thus, the shear modulus, G₃₁, was expressed as a function of 2ε₃₁(=du₁/dx₃+du₃/dx₁) so that an averaged response of

$${\overline{G}}_{13} ( \vert {du}_{1} / {dx}_{3} \vert ) and {\overline{G}}_{31} ( \vert {du}_{3} / {dx}_{1} \vert )$$

was introduced as

$${G}_{31} = \frac{1}{2} \{ {\overline{G}}_{13} ( 2 \vert {ε}_{31} \vert ) + {\overline{G}}_{31} ( 2 \vert {ε}_{31} \vert ) \} . \qquad ( S7 )$$

In the UMAT, the inverse of the elastic compliance matrix (eq. S3) is calculated to make the elastic stiffness matrix (DDSDDE). The stress (STRESS) is updated using the stress and strain at the beginning of the increment, σᵢⱼ (additional state variables) and εᵢⱼ (STRAN), and the increment of strain, Δεᵢⱼ (DSTRAN). The two-dimensional finite element analysis is performed under the plane strain conditions using the UMAT.

### 2.2.2 Validation of the Non-Reciprocal Elastic Model

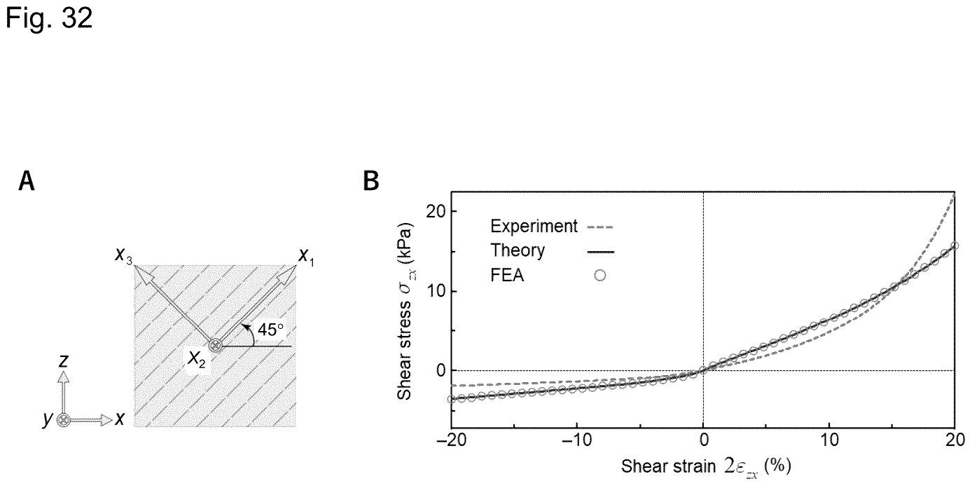

To validate the non-reciprocal elastic model as well as the implementation into the UMAT, the present inventors analyzed the simple shear test of the NR gel under the x-y-z orthonormal coordinate (FIG. 2, A and B). This coordinate was identical to the orthonormal coordinate obtained by the 45° rotation counterclockwise around the x₂-axis from the material's principal directions (FIG. 32A). The response of the shear stress-shear strain, σ_{zx} and 2ε_{zx} (FIG. 32B), showed that the positive and negative directions of 2ε_{zx} (shear right and left) caused the asymmetric response because of the buckling of the nanofillers in the NR gel (FIG. 2B).

The relation between the increments of σ_{zx} and 2ε_{zx} is theoretically derived from eq. S3 by the transformation between the x-y-z and x₁-x₂-x₃ orthonormal coordinates, that is,

$$Δ {σ}_{zx} = \frac{{E}_{1} ( 1 - {υ}_{13} - {υ}_{13} {υ}_{31} ) + {E}_{3} ( 1 - {υ}_{31} )}{4 ( 1 - 2 {υ}_{13} {υ}_{31} )} \times 2 Δ {ε}_{zx} . \qquad ( S8 )$$

The value of ε_{zx} is also decomposed into the components of ε₁₁ and ε₃₃ in the material's principal directions, i.e.,

$${ε}_{11} = - {ε}_{33} = {ε}_{zx} . \qquad ( S9 )$$

Because the values of E₁ and E₃ were modeled as a function of ε₁₁ and ε₃₃ (eqs. S4 and S5) as well as the Poisson's ratios were constants (eq. S6), the shear stress, σ_{zx}, was theoretically calculated as a function of the shear strain, 2ε_{zx}, from eqs. S8 and S9.

In FIG. 32B, the prediction using eqs. S8 and S9 was plotted as the theoretical value. The comparison of the experimental and theoretical values showed the validity of the non-reciprocal elastic model developed here. That reveals that the asymmetry in the positive and negative directions of 2ε_{zx} (shear right and left) results from the tension and compression asymmetries of the responses of E₁ and E₃. In the positive case of 2Δε_{zx}≥0 (shear right), the stiff combination of the Young's moduli, E₁⁺(|ε₁₁|) and E₃⁻(|ε₃₃|), affects the stiff response of σ_{zx}, whereas in the negative case of 2Δε_{zx}<0 (shear left), the soft combination of E₁⁻(|ε₁₁|) and E₃⁺(|ε₃₃|) causes the soft response of σ_{zx}. The developed non-reciprocal elastic model has the ability to describe the mechanical non-reciprocity of the NR gel.

FIG. 32B also demonstrated that the finite element analysis using the UMAT produced the perfectly identical response with the theoretical prediction by eqs. S8 and S9. That indicates the validity of the implementation of the non-reciprocal elastic model.

### 2.2.3 Cylindrical Indentation Analysis of the NR Gel

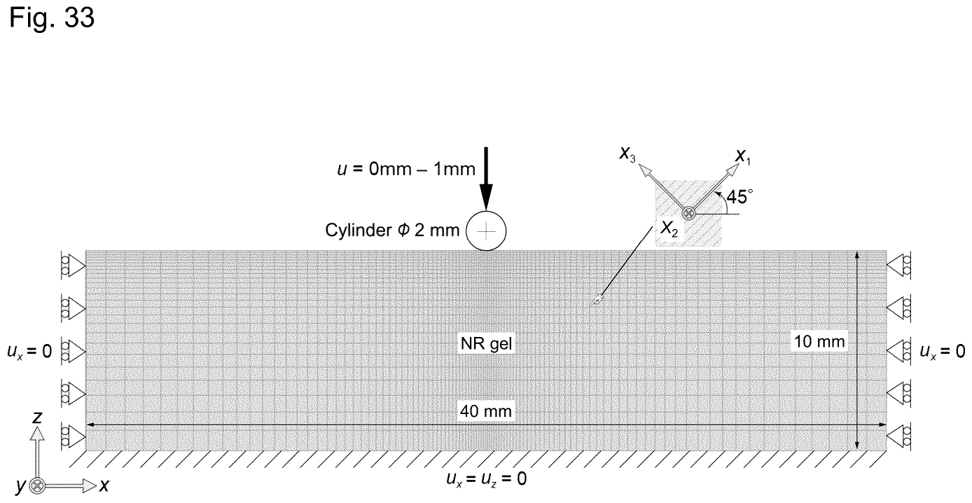

The cylindrical indentation analysis of the NR gel (FIG. 4A) was performed using the two-dimensional finite element model under the plane strain conditions (FIG. 33). The same x-y-z orthonormal coordinate was used to analyze the two-dimensional model, whereas the x₁-x₂-x₃ orthonormal coordinate was embedded in the NR gel to prescribe the material's principal directions. Finite element meshes were prepared using the two-node rigid element R2D2 in the cylinder and the four-node plane strain element CPE4 in the NR gel slab. The number of finite elements was 312 and 2,525 in the cylinder and the NR gel film, respectively. The NR gel slab had zero displacement (uₓ=u_{z}=0) at the bottom face, while uₓ=0 at the side faces. The cylinder was pushed vertically onto the film surface in the depth range 0 mm≤u≤1 mm. The contact surface between the cylinder and the film was assumed to be frictionless.

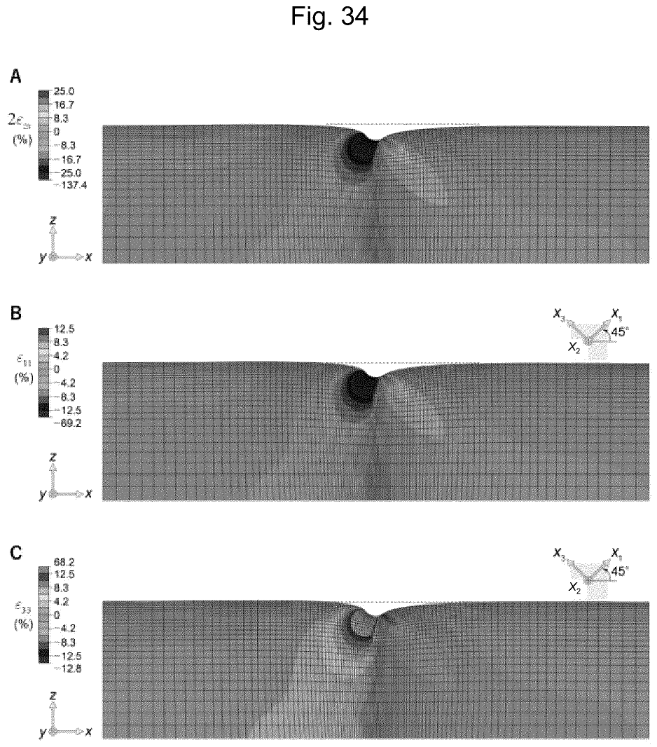

FIG. 34 shows the deformed configuration of the NR gel at u=1 mm as well as the distributions of the shear strain 2ε_{zx} (FIG. 34A) and the axial strains ε₁₁ (FIG. 34B) and ε₃₃ (FIG. 34C), respectively. The comparison of the deformed configuration and the distributions demonstrated that the asymmetric deformation on the surface resulted from the non-reciprocal response in the NR gel slab. Because the shear strain ε_{zx} was decomposed into the components of the axial strains ε₁₁ and ε₃₃ in the material's principal directions (eq. S9), the negative concentration of ε_{zx} (<0) at the left side of the cylinder was consistent with the negative concentration of ε₁₁ (<0) and the positive concentration of ε₃₃ (>0). FIG. 34, B and C show that the buckling of the nanofillers under ε₁₁<0 and ε₃₃ >0 occurred at the large area around the left side of the cylinder. This area caused the highly softer response than the other areas including the right side of the cylinder. The asymmetric deformation on the NR gel was thus theoretically analyzed and understood by the finite element analysis enhanced by the non-reciprocal elastic model.

### 3. Captions for Movies

### Movie S1. Deformation of the Polyacrylamide Gel and the NR Gel in Response to Shear Forces (Movie Time 00:29).

A 10 mm cube of the polyacrylamide gel and that of the NR gel were sheared right (left) with an applied force F₀=0.8 N (−F₀=−0.8 N). The polyacrylamide gel deformed symmetrically, while the NR gel deformed in a highly asymmetric manner.

### Movie S2. Visualization of the Shear Direction-Dependent Buckling of Nanosheets in the NR Gel (Movie Time 00:36).

A 10 mm cube of the NR gel was sheared left and right with the strain of +20% and −20%, respectively. Upon shear to the softer side, nanosheets buckled and reduced their orientation order, so that the light transmittance of the gel was decreased. Upon shear to the harder side, nanosheets hardly buckled, so that the light transmittance of the gel was maintained or even enhanced.

### Movie S3. Directional Transport of Droplets by Using the NR Gel Under Vibration (Movie Time 00:30).

A 2 mm-thick film of the NR gel was pasted at its bottom with a horizontal vibration stage and also pasted at its top with a Teflon plate. Another gel film, directing its harder side oppositely to the previous one, was also set on the vibration stage. When a horizontal symmetric vibration was applied from the stage, the water droplets loaded on the Teflon plates drifted toward the harder sides of the NR gel films. This unidirectional transport continuously took place when water droplets were continuously loaded.

### Movie S4. Anti-Gravity Transport of Droplets by Using the NR Gel Under Vibration (Movie Time 00:34).

A 2 mm-thick film of the NR gel was pasted at its one side with a vertical vibration stage and pasted at the other side with a Teflon plate. When a vertical symmetric vibration was applied from the stage, amazingly, the water droplets unidirectionally climbed up the vertical Teflon plate against gravity.

### Movie S5. Directional Rotation of a Wheel by the NR Gel Under Vibration (Movie Time 01:06).

A 2 mm-thick disk of the NR gel, composed of ⅙ sector-shaped gel films with arranging their harder directions clockwise, was pasted at its bottom with a horizontal vibration stage and pasted at its top with a Teflon dish to act as the rim for the rotating wheel. A Teflon disk was placed onto the Teflon dish to act as the wheel. When a horizontal symmetric vibration was applied from the stage, the wheel rotated clockwise at a constant velocity. When the arrangement of the harder directions in the gel disk was reversed to counterclockwise, the rotation direction of the wheel was reversed, accordingly. This rotation was quite stable and tolerant to external mechanical disturbances.

Movie S6. Energy Harvest from Environmental Vibrations by Using the NR Gel (Movie Time 00:55).

A 2 mm-thick disk of the NR gel, composed of ⅙ sector-shaped gel films with arranging their harder directions clockwise, was loaded with a Teflon rim and a Teflon wheel, and then placed on an oil pump or a wireless speaker. When the pump or the speaker started working, the wheel started rotation at a constant velocity. Thus, tiny environmental vibration was converted into a directional mechanical energy that drove the wheel to rotate.

### Movie S7. Deformation of R Gel and NR Gel Upon Local Compression (Movie Time 00:22).

A 10 mm-thick slab of the R gel and that of the NR gel were locally compressed by a q 2 mm cylinder that laid parallel to the surface of the gel slab with the indentation depth up to 2 mm. The R gel deformed symmetrically, while the NR gel deformed in a highly asymmetric manner.

### Movie S8. Theoretical Prediction for the Deformation Profiles of the NR Gel Upon Local Compression (Movie Time 00:06).

The deformation profile of a 10 mm-thick slab of the NR gel upon the indentation of a q 2 mm cylinder was theoretically predicted by using the finite element analysis, where the asymmetry in the outer shape and strain distribution was confirmed.

### Movie S9. Directional Bouncing of Free-Dropped Balls on the NR Gel (Movie Time 00:50).

A q 2 mm metal ball was released from an electromagnet above a 2 mm-thick gel film and allowed to free-drop onto the gel film. The R gel bounced back the ball vertically, while the NR gel always bounced the ball towards its harder side.

Movie S10. Directional Migration of C. elegans on NR Gels (Movie Time 00:46).

A group of C. elegans was transferred to a gel film, and their migration was observed. Surprisingly, in the case of the NR gel, all worms migrated to the harder side and finally gathered at the harder end of the gel film. On the contrary, in a control experiment using the R gel, C. elegans exhibited a totally random migration.

### REFERENCES

1. R. J. Potton, Rep. Prog. Phys. 67, 717-754 (2004).
2. D. L. Sounas, A. Alu, Nat. Photon. 11, 774-783 (2017).
3. L. Feng et al., Science 333, 729-733 (2011).
4. H. Xu, L. Jiang, A. A. Clerk, J. G. E. Harris, Nature 568, 65-69 (2019).
5. R. Fleury, D. L. Sounas, C. F. Sieck, M. R. Haberman, A. Alu, Science 343, 516-519 (2014).
6. L. Shao et al., Nat. Electron. 3, 267-272 (2020).
7. C. Rasmussen, L. Quan, A. Alu, J. Appl. Phys. 129, 210903 (2021).
8. V. Peano, M. Houde, F. Marquardt, A. A. Clerk, Phys. Rev. X 6, 041026 (2016).
9. H. K. Lau, A. A. Clerk, Nat. Commun. 9, 4320 (2018).
10. C. Coulais, D. Sounas, A. Alu, Nature 542, 461-464 (2017).
11. H. Nassar et al., Nat. Rev. Mater. 5, 667-685 (2020).
12. D. L. Sounas, J. Soric, A. Alu, Nat. Electron. 1, 113-119 (2018).
13. Y. Wang et al., Phys. Rev. Lett. 121, 194301 (2018).
14. L. M. Nash et al., Proc. Natl. Acad. Sci. USA 112, 14495-14500 (2015).
15. M. Brandenburger et al., Nat. Commun. 10, 4608 (2019).
16. G. Librandi, E. Tubaldi, K. Bertoldi, Nat. Commun. 12, 3454 (2021).
17. M. Shaat, Sci. Rep. 10, 21676 (2020).
18. M. Shaat, M. A. Moubarez, M. O. Khan, M. A. Khan, A. Alzo′ubi, Phys. Rev. Appl. 14, 014005 (2020).
19. R. M. Jones, Buckling of Bars, Plates, and Shells (Bull Ridge Publishing, 2006).
20. N. Hu, R. Burgueno, Smart Mater. Struct. 24, 063001 (2015).
21. A. V. Thomas et al., Adv. Mater. 27, 3256-3265 (2015).
22. S. Deng, V. Berry, Mater. Today 19, 197-212 (2016).
23. M. Morozov, O. Manor, Curr. Opin. Colloid Interface Sci. 36, 35-37 (2018).
24. N. A. Croll, J. Zool. 176, 159-176 (1975).
25. P. M. Meneely, C. L. Dahlberg, J. K. Rose, Curr. Protoc. Essent. Lab. Tech. 19, e35 (2019).
26. T. Sasaki et al., Chem. Mater. 10, 4123-4128 (1998).
27. K. Akatsuka et al., J. Phys. Chem. C 116, 12426-12433 (2012).
28. E. Tampakakis et al., Nat. Protoc. 3, 1925-1931 (2008).
29. X. Lu et al. Proc. Nat 5 1. Acad. Sci. U.S.A. 114, E₉₇₉₃-E₉₈₀₁ (2017).
30. http://imagej.nih.gov/ij/
31. https://www.rigaku.com/support/software/2DP
32. K. Sano et al., Nat. Commun. 12, 6771 (2021).
33. S. Brenner, Genetics 77, 71-94 (1974).
34. A. Javer et al., Nat. Methods 15, 645-646 (2018).
35. E. E. Charrier et al., APL Bioeng. 4, 036104 (2020).
36. J. Liu et al., Macromol. Rapid Commun. 34, 1002 (2013).
37. Z. M. Xia et al., Comp. Mater. Sci. 150, 252 (2018).
38. ABAQUS 6.14 User Documentation (Dassault Systems SIMULIA Corporation, 2014).
39. P. Vannucci, Anisotropic Elasticity (Springer, Nature Singapore, 2018).
40. S. Toki, B. S. Hsiao, “Deformation-Induced Structure Changes in Elastomeric Nanocomposites” in Recent Advances in Elastomeric Nanocomposites. Advanced Structured Materials, V. Mittal, J. Kim, K. Pal, Eds. (Springer, Berlin, Heidelberg, 2011), vol 9. pp. 135-154.
41. G. A. Evingur, O. Pekcan, Polym. Bull. 75, 1431-1439 (2018).
42. J. Shen et al., Composites: Part A 43, 1476-1481 (2012).
43. H. Cong, P. Wang, S. Yu, Small 10, 448-453 (2014).
44. R. Liu et al., J. Mater. Chem. 22, 14160-14167 (2012).

1. A gel structure showing an asymmetric response to mechanical force.

2. The gel structure according to claim 1, which is a hydrogel.

3. The gel structure according to claim 2, wherein the hydrogel contains nanosheets.

4. The gel structure according to claim 3, wherein the nanosheets are oriented so as to tilt 15° to 75° clockwise from a direction of gravity, as viewed from a side of the gel.

5. The gel structure according to claim 3, wherein the nanosheets are graphene oxide nanosheets, niobium oxide nanosheets, titanium oxide nanosheets, clay nanosheets, alumina nanotubes, or carbon nanotubes.

6. The gel structure according to claim 1, wherein the mechanical force is a shear force or vibration.

7. The gel structure according to claim 6, wherein when a bottom face of the gel structure is fixed to a floor and a left or right shear force is applied to a top face thereof, a difference in elastic modulus between both is 10 times or more.

8. A method for producing the gel structure according to claim 1, comprising changing a direction of a magnetic field such that the nanosheets are contained in a tilted state in the hydrogel.

9. The method according to claim 8, comprising placing a pre-gel solution including a monomer, a crosslinking agent, and graphene oxide nanosheets, niobium oxide nanosheets, titanium oxide nanosheets, clay nanosheets, alumina nanotubes, or carbon nanotubes in a container, and applying a magnetic field to the container while tilting the container, thereby polymerizing the monomer.

10. The gel structure according to claim 1, which is a gel structure showing an asymmetric response to mechanical force, and comprises a gel composition including at least one type of anisotropic nanostructures, at least one type of liquid, and at least one type of polymer having a crosslinked structure.

11. The gel structure according to claim 10, wherein the liquid is water.

12. The gel structure according to claim 10, wherein the plurality of at least one type of anisotropic nanostructures is selected from the group consisting of graphene oxide nanosheets, niobium oxide nanosheets, titanium oxide nanosheets, clay nanosheets, alumina nanotubes, carbon nanotubes, and nanochannel pores.

13. The gel structure according to claim 10, which has at least one surface in a stationary state, and in which longitudinal axes of the at least one type of anisotropic nanostructures are oriented approximately parallel to each other and non-parallel and non-perpendicular to the at least one surface.

14. A gel structure comprising a gel composition including at least one type of anisotropic nanostructures, at least one type of liquid, and at least one type of polymer having a crosslinked structure, which has at least one surface in a stationary state, and in which longitudinal axes of the at least one type of anisotropic nanostructures are oriented approximately parallel to each other and non-parallel and non-perpendicular to the at least one surface.

15. The gel structure according to claim 10, which exhibits elastic moduli of different magnitudes when shear forces of the same magnitude are applied in a first direction and a second direction different from the first direction in a plane parallel to the one surface.

16. The gel structure according to claim 10, which is in a film form.

17. The gel structure according to claim 10, which is used as a culture medium for cells, microorganisms, or nematodes.

18. A method for producing a gel structure, comprising:
a first step of accommodating a gel raw material composition, which includes at least one type of anisotropic nanostructures, at least one type of liquid, and a polymerizable composition, in a container having at least one identifiable face; and
a second step of carrying out a polymerization reaction of the polymerizable composition in the container,
wherein in the second step, the at least one type of anisotropic nanostructures is oriented with their longitudinal axes approximately parallel to each other and non-parallel and non-perpendicular to the at least one identifiable face.

19. The method according to claim 18, wherein in the second step, at least one type of physical stimulus is applied to the gel raw material composition.

20. The method according to claim 18, wherein the polymerizable composition includes at least one type of monomer, at least one type of crosslinking agent, and at least one type of polymerization initiator.
